(19) United States
(12) Patent Application Publication
(10) Pub. No.: US 2019/0066827 A1
(43) Pub. Date: Feb. 28, 2019

(54) MEDICAL INFORMATION PROCESSING SYSTEM

(71) Applicant: Canon Medical Systems Corporation, Otawara-shi (JP)

(72) Inventors: Kazumasa NORO, Shioya-gun (JP); Yusuke KANO, Nasushiobara (JP); Kazuki UTSUNOMIYA, Nasushiobara (JP); Longxun PIAO, Nasushiobara (JP)

(73) Assignee: Canon Medical Systems Corporation, Otawara-shi (JP)

(21) Appl. No.: 16/110,810

(22) Filed: Aug. 23, 2018

(30) Foreign Application Priority Data
Aug. 24, 2017 (JP) 2017-161504

(51) Int. Cl. G16H 15/00; G06F 3/0482; G16H 30/20

(52) U.S. Cl. CPC G16H 15/00; G06F 3/0486; G16H 30/20; G06F 3/0482

(57) ABSTRACT

A medical information processing system comprises a memory and processing circuitry. The memory is configured to store therein pieces of first medical data each of which includes at least image data indicating medical information and pieces of second medical data each of which is included in data indicating medical information and is other than the first medical data, so that the pieces of first medical data and the pieces of second medical data are associated with times. The processing circuitry is configured to receive an operation performed on any of the pieces of second medical data displayed in time series, to acquire a time range corresponding to the received operation, extract at least one of the pieces of first medical data corresponding to the time range from the memory, and display at least one extracted piece of first medical data.

### CROSS-REFERENCE TO RELATED APPLICATIONS

This application is based upon and claims the benefit of priority from Japanese Patent Application No. 2017-161504, filed on Aug. 24, 2017; the entire contents of which are incorporated herein by reference.

### FIELD

Embodiments described herein relate generally to a medical information processing system.

### BACKGROUND

Doctors make diagnoses and provide treatments by comprehensively using various types of data indicating medical information. For example, doctors make diagnoses and provide treatments while comparing image data (e.g., ultrasonic image data, X-ray image data, image interpretation reports) indicating medical information, with data other than image data (e.g., prescription records, vital data). For example, during treatment for heart failure, doctors recognize the state of the heart failure (e.g., whether the heart failure is acute or chronic) on the basis of ultrasonic image data acquired of the heart of the patient and adjust the types and the doses of medications in accordance with prescription records and the state of the heart failure.

In this situation, a method (a time-series display method) is known by which pieces of image data and prescription records are displayed in time series. However, when the pieces of image data are displayed in time series, because there is no choice but displaying the individual pieces of image data in reduced size in thumbnails or the like, it is difficult to observe the image data when the pieces of image data are simply displayed in time series. It therefore requires long time to make a comparison in time series between the pieces of image data and the prescription records. For example, when wishing to check advantageous effects of a medication by using image data, doctors and nurses themselves need to search for a piece of image data corresponding to the time when the medication was started and another piece of image data corresponding to the time when the medication was ended.

### BRIEF DESCRIPTION OF THE DRAWINGS

FIG. 1 is a block diagram illustrating an exemplary configuration of a medical information processing system according to a first embodiment;

FIG. 2A is a table illustrating an example of prescription records according to the first embodiment;

FIG. 2B is a table for explaining displaying prescription records in time series according to the first embodiment;

FIG. 2C is another table for explaining displaying prescription records in time series according to the first embodiment;

FIG. 2D is a drawing illustrating an example of displaying prescription records in time series according to the first embodiment;

FIG. 3 is a chart illustrating an example of a time range according to the first embodiment;

FIG. 4A is a drawing for explaining a time range acquiring process according to the first embodiment;

FIG. 4B is a table for explaining the time range acquiring process according to the first embodiment;

FIG. 4C is another table for explaining the time range acquiring process according to the first embodiment;

FIG. 4D is yet another table for explaining the time range acquiring process according to the first embodiment;

FIG. 5 is a drawing for explaining a medical image data extracting process according to the first embodiment;

FIG. 6 is a drawing for explaining a medical image data display process according to the first embodiment;

FIG. 7 is another drawing for explaining the medical image data extracting process according to the first embodiment;

FIG. 8 is another drawing for explaining the medical image data display process according to the first embodiment;

FIG. 9 is a flowchart for explaining a flow in a series of processes performed by the medical information processing system according to the first embodiment;

FIG. 10 is a drawing illustrating another example of displaying prescription records in time series according to the first embodiment;

FIG. 11 is yet another table for explaining the time range acquiring process according to the first embodiment;

FIG. 12 is a drawing for explaining displaying medical image data in time series according to a second embodiment;

FIG. 13A is a table for explaining a time range acquiring process according to the second embodiment;

FIG. 13B is a table for explaining a time range display process according to the second embodiment;

FIG. 14 is a drawing for explaining the time range display process according to the second embodiment;

FIG. 15 is a flowchart for explaining a flow in a series of processes performed by a medical information processing system according to the second embodiment; and

FIG. 16 is a drawing for explaining a report display process according to a third embodiment.

### DETAILED DESCRIPTION

A medical information processing system comprises a memory and processing circuitry. The memory is configured to store therein pieces of first medical data each of which includes at least image data indicating medical information and pieces of second medical data each of which is included in data indicating medical information and is other than the first medical data, so that the pieces of first medical data and the pieces of second medical data are associated with times. The processing circuitry is configured to receive an operation performed on any of the pieces of second medical data displayed in time series, to acquire a time range corresponding to the received operation, extract at least one of the pieces of first medical data corresponding to the time range from the memory, and display at least one extracted piece of first medical data.

Exemplary embodiments of the medical information processing system will be explained in detail, with reference to the accompanying drawings.

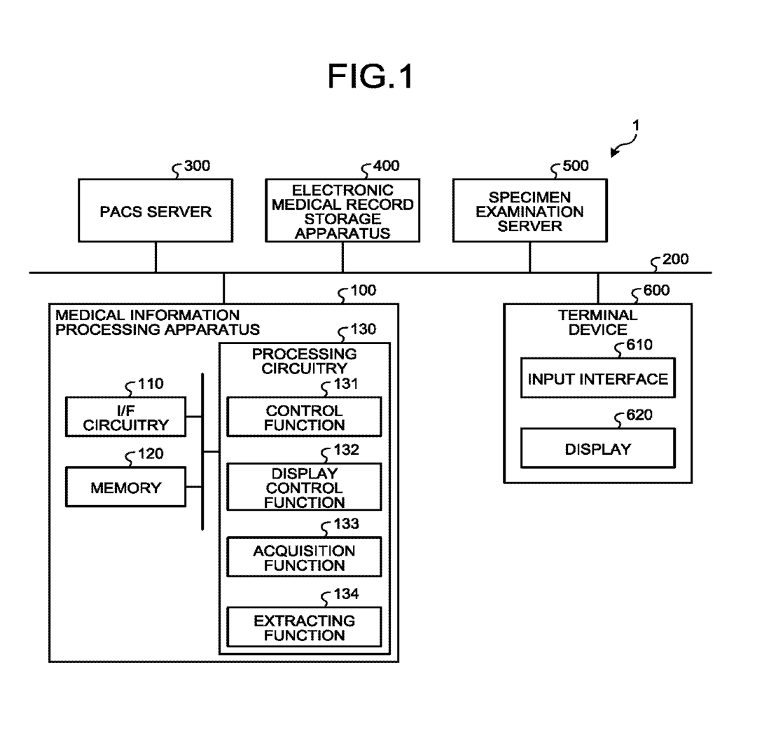

FIG. 1 is a block diagram illustrating an exemplary configuration of a medical information processing system 1 according to a first embodiment. As illustrated in FIG. 1, the medical information processing system 1 includes a medical information processing apparatus 100, a network 200, a Picture Archiving and Communication System (PACS) server 300, an electronic medical record storage apparatus 400, a specimen examination server 500, and a terminal device 600.

As illustrated in FIG. 1, the medical information processing apparatus 100 is connected, via the network 200, to the PACS server 300, the electronic medical record storage apparatus 400, the specimen examination server 500, and the terminal device 600 so as to be able to communicate therewith. For example, the medical information processing apparatus 100, the PACS server 300, the electronic medical record storage apparatus 400, and the specimen examination server 500 are installed in a hospital and are connected to one another via the network 200 realized by an intra-hospital Local Area Network (LAN) or the like. Further, for example, the terminal device 600 is realized by using a personal computer or a tablet or the like that can be carried around by an operator such as a doctor, a nurse, or the like and is connected, via the network 200, to the medical information processing apparatus 100, the PACS server 300, the electronic medical record storage apparatus 400, and the specimen examination server 500 so as to be able to communicate therewith.

The PACS server 300 is an apparatus configured to store therein various types of image data such as medical image data acquired by medical image diagnosis apparatuses (not illustrated). For example, the PACS server 300 is configured to receive X-ray image data acquired by an X-ray diagnosis apparatus (not illustrated) and to store therein the X-ray image data so as to be associated with the time at which the X-ray image data was acquired and a patient ID. Further, for example, the PACS server 300 is configured to receive ultrasonic image data acquired by an ultrasonic diagnosis apparatus (not illustrated) and to store therein the ultrasonic image data so as to be associated with the time at which the ultrasonic image data was acquired and a patient ID.

For example, the PACS server 300 stores the medical image data therein in a Digital Imaging and Communications in Medicine (DICOM) format. In that situation, for example, with respect to each of the acquired pieces of medical image data, the PACS server 300 records the date on which the medical image data was acquired into a tag “(0008,0022) Acquisition Date” in the DICOM system, records the time at which the medical image data was acquired into a tag “(0008,0032) Acquisition Time”, and records the patient ID into a tag “(0010,0020) Patient ID”. In other words, the PACS server 300 is configured to store therein the pieces of medical image data so as to be associated with the times.

Further, the PACS server 300 is capable of storing the medical image data therein as still images or as moving images. In one example, the PACS server 3070 stores the medical image data therein as still image data in a JPEG format, a PNG format, a BMP format, or the like. Further, as another example, the PACS server 300 stores the medical image data therein as moving image data in an MP4 format, an MOV format, an MPEG format, or the like.

Further, the PACS server 300 is configured to store therein various types of reports. For example, as image data, the PACS server 300 stores therein image interpretation reports of medical image data, care providing reports created by nurses, medical examination reports, and the like so as to be associated with times at which reports were created and patient IDs. In this situation, examples of the medical examination reports include a specimen report related to a specimen (e.g., blood) collected from a patient, a bacteria report related to bacteria in a patient, and a physiological report related to physiological information of a patient. For example, the PACS server 300 stores the reports therein as image data in a PDF format, a JPEG format, or the like.

For example, when the PACS has been introduced to the hospital, the PACS server 300 is installed in the hospital as an image server in the PACS. For example, the PACS server 300 is realised by using a computer device such as a Database (DB) server and is configured to store the image data into a semiconductor memory element such as a Random Access Memory (RAM), a flash memory, or the like, or a storage circuit such as a hard disk, an optical disk, or the like. The PACS server 300 is an example of the memory. Further, the medical image data and the reports stored by the PACS server 300 is an example of the image data indicating medical information.

In the first embodiment, the image data indicating medical information will be explained as an example of the first medical data; however, possible embodiments are not limited to this example. In other words, the PACS server 300 is configured to store therein the first medical data including at least image data indicating medical information so as to be associated with times.

The electronic medical record storage apparatus 400 is an apparatus configured to store therein medical data related to various types of diagnosis and treatment performed in the hospital. For example, the electronic medical record storage apparatus 400 stores therein data (vital data) measured from patients such as pulse counts, heartbeat counts, breathing rates, blood pressure values, body temperatures, percutaneous arterial blood oxygen saturation (SpO2) values, and the like, so as to be associated with the times at which the data was measured and patient IDs. Further, for example, the electronic medical record storage apparatus 400 stores therein records of prescription for patients so as to be associated with medication times and patient IDs. In other words, the electronic medical record storage apparatus 400 is configured to tore therein the medical data so as to be associated with the times.

For example, the electronic medical record storage apparatus 400 is tailed as a part of an electronic medical record system introduced to the hospital and is configured to store therein medical data generated by the electronic medical record system. For example, the electronic medical record storage apparatus 400 is realized by using a computer device such as a database (DB) server and is configured to store the medical data into a semiconductor memory element such as a RAM, a flash memory, or the like, or a storage circuit such as a hard disk, an optical disk, or the like. The electronic medical record storage apparatus 400 is an example of the memory. The medical data stored by the electronic medical record storage apparatus 400 is an example of the data (non-image data) that is included in data indicating medical information and is other than image data.

The specimen examination server 500 is an apparatus configured to store therein examination data of specimens collected from patients. For example, the specimen examination server 500 stores therein examination data (e.g., blood cell counts, ion concentration levels, etc.) of blood samples collected from patients so as to be associated with times. In one example, the specimen examination server 500 stores the examination data therein so as to be associated with the times at which the specimens were collected from the patients or the times at which the examination data was measured, as well as patient IDs. In other words, the specimen examination server 500 is configured to store therein the examination data so as to be associated with the times.

For example, the specimen examination server 500 is installed as a part of a specimen examination system introduced to the hospital and is configured to store therein examination data in the specimen examination system. For example, the specimen examination server 500 is realized by using a computer device such as a database (DB) server and is configured to store the examination data into a semiconductor memory element such as a RAM, a flash memory, or the like, or a storage circuit such as a hard disk, an optical disk, or the like. The specimen examination server 500 is an example of the memory. Further, the examination data stored by the specimen examination server 500 is an example of non-image data.

The non-image data is an example of the second medical data, which is the data that is included in data indicating medical information and is other than the first medical data. In this situation, when the first medical data is image data, the second medical data is non-image data. In other words, the electronic medical record storage apparatus 400 and the specimen examination server 500 are each configured to store therein the second medical data, which is the data that is included in data indicating medical information and is other than the first medical data, so as to be associated with the times.

As examples of the image data, the medical image data and the reports are explained above; however, possible embodiments are not limited these examples. For instance, electrocardiograms are records of electric activities of the heart. In some situations, numerical values of electrocardiograms are stored as non-image data, but in other situations, charts thereof are stored as image data. Further, as examples of the non-image data, the medical data and the examination data are explained above; however, possible embodiments are not limited to these examples. For instance, besides the medical data and the examination data, examples of the non-image data include measured data (e.g., blood vessel diameters, blood flow volumes, etc.) of images acquired from patients.

The medical information processing apparatus 100 is configured to acquire the image data and the non-image data via the network 200 and to perform various types of information processing processes by using the acquired image data and non-image data. For example, the medical information processing apparatus 100 is realized by using a computer device such as a workstation.

In the following sections, as an example, a situation will be explained in which the medical information processing apparatus 100 acquires medical image data as the image data. Also, in the following sections, as an example, a situation will be explained in which the medical information processing apparatus 100 acquires prescription records as the non-image data.

As illustrated in FIG. 1, the medical information processing apparatus 100 includes interface (I/F) circuitry 110, a memory 120, and processing circuitry 130.

The I/F circuitry 110 is connected to the processing circuitry 130 and is configured to control transfer and communication of various types of data to and from external apparatuses (the PACS server 300, the electronic medical record storage apparatus 400, the specimen examination server 500, the terminal device 600, etc.) provided on the outside of the medical information processing apparatus 100. For example, the I/F circuitry 110 is configured to receive the medical image data from the PACS server 300 and to output the received medical image data to the processing circuitry 130. Further, for example, the I/F circuitry 110 is configured to receive a prescription record from the electronic medical record storage apparatus 400 and to output the received prescription record to the processing circuitry 130. For example, the I/F circuitry 110 is realized by using a network card, a network adaptor, a Network Interface Controller (NIC), or the like.

The memory 120 is connected to the processing circuitry 130 and is configured to store various types of data therein. For example, the memory 120 is configured to store therein the medical image data received from the PACS server 300 and the prescription records received from the electronic medical record storage apparatus 400. For example, the memory 120 is realized by using a semiconductor memory element such as a Random Access Memory (RAM), a flash memory, or the like, or a hard disk, an optical disk, or the like.

The processing circuitry 130 is configured to control overall processes performed by the medical information processing apparatus 100 by executing a control function 131, a display control function 132, an acquisition function 133, and an extracting function 134. For example, the processing circuitry 130 is realized by using a processor.

For example, by reading and executing a computer program (hereinafter, simply “program”) corresponding to the control function 131 from the memory 120, the processing circuitry 130 stores the medical image data and the prescription record output from the I/F circuitry 110 into the memory 120. Further, for example, by reading and executing a program corresponding to the display control function 132 from the memory 120, the processing circuitry 130 exercises display control. For example, the display control function 132 reads the prescription records stored in the memory 120 and displays the read prescription records in time series on a display 620 of the terminal device 600. Further, for example, by reading and executing programs corresponding to the display control function 132, the acquisition function 133, and the extracting function 134 from the memory 120, the processing circuitry 130 acquires a time range corresponding to an operation performed on any of the prescription records displayed in time series and further displays one or more pieces of medical image data corresponding to the time range on the display 620. The process of acquiring the time range and the process of displaying the pieces of medical image data corresponding to the time range will be explained later.

In this situation, the display control function 132 is an example of the display control unit. The acquisition function 133 is an example of the acquiring unit. The extracting function 134 is an example of the extracting unit.

As illustrated in FIG. 1, the terminal device 600 includes an input interface 610 and the display 620. The input interface 610 is configured to convert an input operation received from the operator into an electrical signal and to output the electrical signal to the medical information processing apparatus 100. For example, the input interface 610 is realized by using a trackball, a switch button, a mouse, a keyboard, a touch panel, and/or the like.

The display 620 is configured to display various types of data output from the medical information processing apparatus 100. For example, the display 620 is realized by using a liquid crystal monitor, a Cathode Ray Tube (CRT) monitor, a touch panel, or the like. The input interface 610 and the display 620 may be integrated together. For example, the input interface 610 and the display 620 may be realized by using a touch panel.

An overall configuration of the medical information processing system 1 according to the first embodiment has thus been explained. The medical information processing system 1 according to the first embodiment structured as described above is configured to make it easy to compare the image data with the non-image data. More specifically, with the processes performed by the processing circuitry 130 as explained in detail below, the medical information processing system makes it easy to compare the image data with the non-image data, by acquiring a time range corresponding to an operation performed on any of the pieces of non-image data displayed in time series and to display at least one of the pieces of image data corresponding to the time range. In the following sections, processes performed by the medical information processing system 1 according to the first embodiment will be explained in detail.

First, the display control function 132 displays the prescription records in time series. For example, at first, the I/F circuitry 110 receives the prescription records from the electronic medical record rage apparatus 400 and further outputs the received prescription records to the processing circuitry 130. Subsequently, the control function 131 stores the prescription records output from the I/F circuitry 110 into the memory 120. After that, the display control function 132 reads the prescription records from the memory 120 and displays the read prescription records on the display 620 in time series.

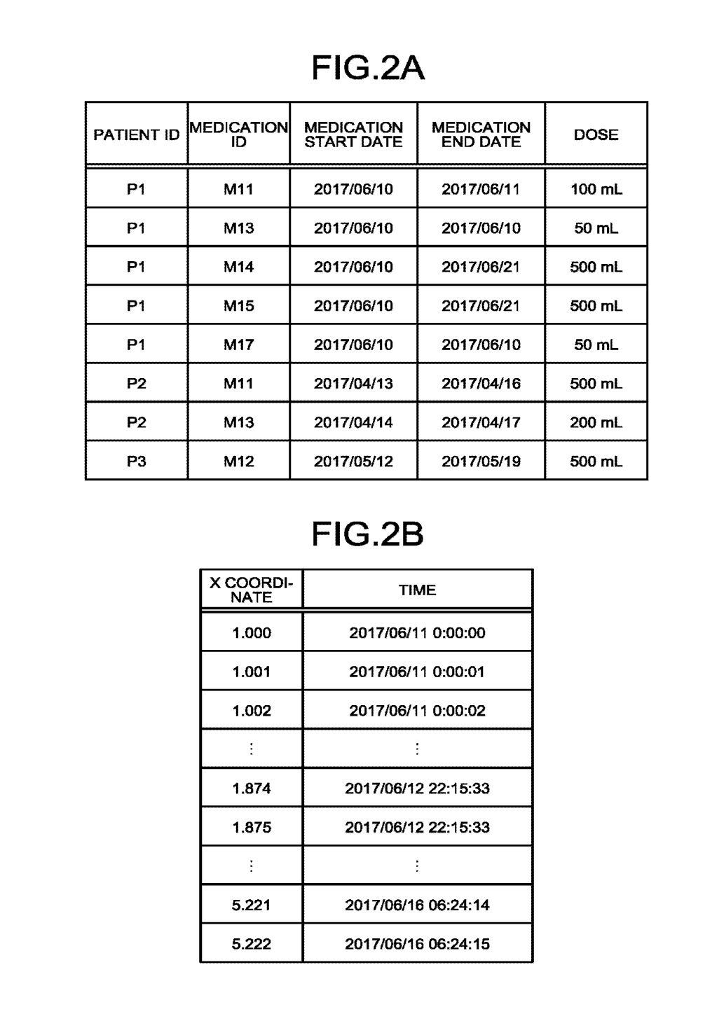

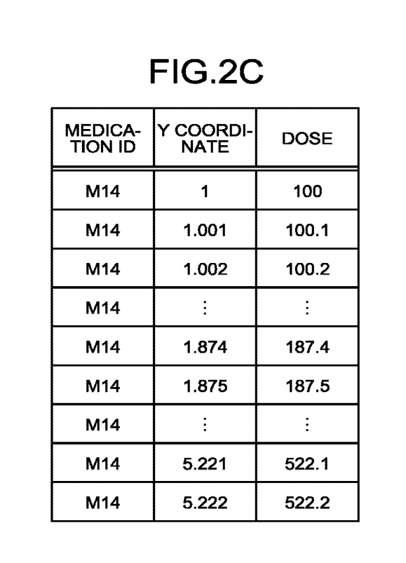

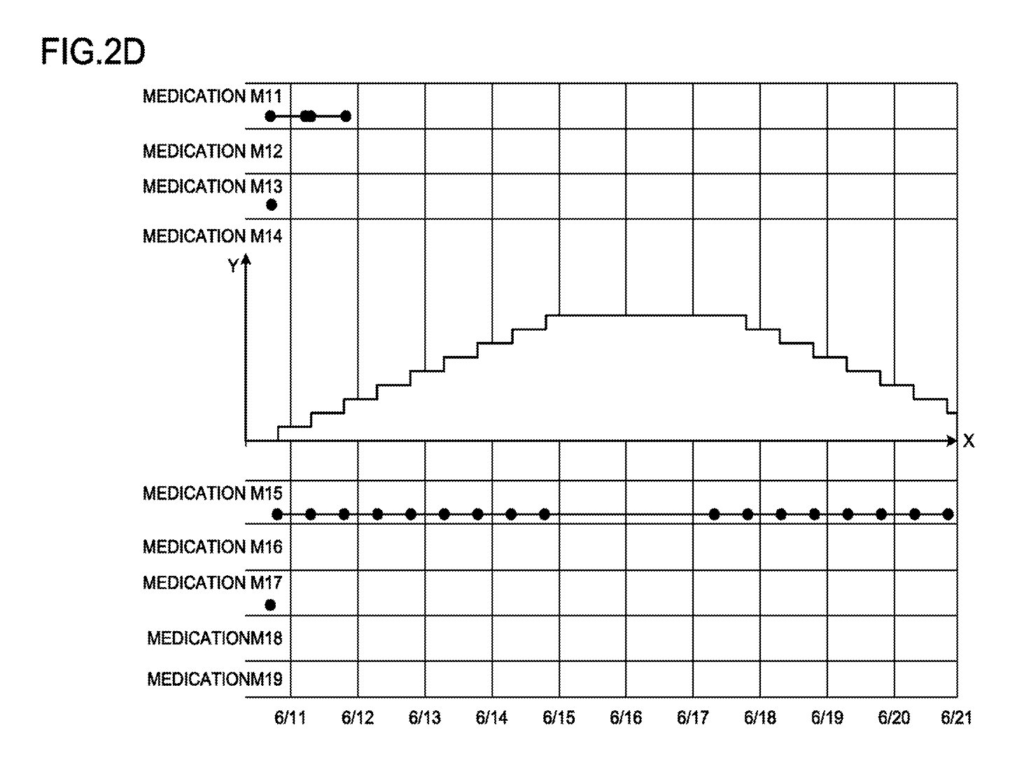

In this situation, an example of the prescription records stored in the electronic medical record storage apparatus 400 will be explained, with reference to FIG. 2A. FIG. 2A is a table illustrating an example of the prescription records according to the first embodiment.

For example, as illustrated in FIG. 2A, as the prescription records, the electronic medical record storage apparatus 400 stores therein items such as “patient IDs”, “medication IDs”, “medication start dates”, “medication end dates”, and “dose”, so as to be associated one another. In this situation, the “patient IDs” are each a piece of information for identifying the patient to whom the medication was administered. The “medication IDs” are each a piece of information for identifying the type of the medication. The “medication start dates” are each the date on which the medication was started. The “medication end dates” are each the date on which the medication was ended. When the medication is continuing, the “medication end date” may be the current date. Further, the “dose” each denotes a total amount of the medication.

For example, from a doctor, a nurse, or the like, who administered medication, the electronic medical record storage apparatus 400 receives and stores therein, as a prescription record, an input of items such as a “patient ID”, a “medication ID”, a “medication start date”, a “medication end date”, and an “dose”. Further, the electronic medical record storage apparatus 400 is also capable of storing therein the input prescription record so as to be associated with the time of the input.

More specifically, as illustrated in FIG. 2A, the electronic medical record storage apparatus 400 stores therein, as a prescription record, information indicating that in the time period from “2017 Jun. 10” to “2017 Jun. 11”, “100 mL” of a medication M11 was administered to patient P1. Further, the electronic medical record storage apparatus 400 stores therein, as another prescription record, information indicating that on “2017 Jun. 10”, “50 mL” of a medication M13 was administered to patient P1. In addition, the electronic medical record storage apparatus 400 stores therein, as yet another prescription record, information indicating that in the time period from “2017 Jun. 10” to “2017 Jun. 21”, “500 mL” of a medication M14 was administered to patient P1. Also, the electronic medical record storage apparatus 400 stores therein, as yet another prescription record, information indicating that in the time period from “2017 Jun. 10” to “2017 Jun. 21”, “500 mL” of a medication M15 was administered to patient P1. Additionally, the electronic medical record storage apparatus 400 stores therein, as yet another prescription record, information indicating that on “2017 Jun. 10”, “50 mL” of a medication M17 was administered to patient P1.

Further, the electronic medical record age apparatus 400 stores therein, as yet another prescription record, information indicating that in the time period from “2017 Apr. 13” to “2017 Apr. 16”, “500 mL” of the medication M11 was administered to patient P2. Further, the electronic medical record storage apparatus 400 stores therein, as yet another prescription record, information indicating that in the time period from “2017 Apr. 14” to “2017 Apr. 17”, “200 mL” of the medication M13 was administered to patient P2. In addition, the electronic medical record storage apparatus 400 stores therein, as yet another prescription record, information indicating that in the time period from “2017 May 12” to “2017 May 19”, “500 mL” of a medication was administered to patient P3.

Although FIG. 2A illustrates only the “medication start dates” and the “medication end dates”, the electronic medical record storage apparatus 400 may store therein the prescription records so as to be associated with more detailed times. For instance, the electronic medical record storage apparatus 400 may be configured to store therein, while using the medication administration at each time as a unit, the patient ID of the patient to whom the medication was administered, the medication ID of the administered medication, the date and the time of the administration, and the dose, so as to be associated one another.

The I/F circuitry 110 receives the prescription records from the electronic medical record storage apparatus 400 and outputs the received prescription records to the processing circuitry 130. Subsequently, the control function 131 stores the prescription records output from/F circuitry 110 into the memory 120. In this situation, similarly to the electronic medical record storage apparatus 400, the memory 120 stores the prescription records therein so as to be associated with the times. Further, the display control function 132 reads the prescription records from the memory 120 and displays the read prescription records in time series on the display 620.

For example, the display control function 132 at first receives, from the operator, a selecting operation to select a patient and a medication, via the input interface 610. In the following sections, an example will be explained in which the patient P1 is selected as the patient and the medication M14 is selected as the medication. Subsequently, the display control function 132 displays the doses of the medication M14 to the patient P1 in time series. In other words, the display control function 132 is configured to display, as the prescription records, the doses of the selected medication to the selected patient in time series. In one example, the display control function 132 displays a chart plotting the doses of the medication 1014 to the patient P1 by using a graph of which the horizontal axis expresses time, whereas the vertical axis expresses doses.

More specifically, the display control function 132, at first, sets the time axis (the X-axis) according to the medication start date and the medication end date of the medication M14 to the patient. P1. For example, the display control function 132 sets a correspondence relationship between each of the coordinates (the X coordinates) on the display screen and the times, as illustrated in a coordinate/time conversion table in FIG. 2B. Further, the display control function 132 sets the axis (the Y-axis) indicating the doses according to the type of the displayed medication or the like. For example, the display control function 132 sets a correspondence relationship between the type (the medication ID) of the displayed medication, each of the coordinates (the Y coordinates) on the display screen, and the doses, as illustrated in a coordinate/dose conversion table in FIG. 22. FIGS. 2B and 2C are drawings for explaining displaying the prescription records in time series according to the first embodiment.

After that, the display control function 132 displays the doses of the medication M14 to the patient P1 in time series as illustrated in FIG. 2D, by plotting the administered times and the doses of the medication M14 to the patient P1 according to the X coordinates illustrated in FIG. 2P and the Y coordinates illustrated in FIG. 22. FIG. 2D is a drawing illustrating an example of the displaying of the prescription records in time series according to the first embodiment.

The bar graph in FIG. 29 displays the doses of the medication M14 to the patient. P1 in time series, for the time period from the medication start date “2017 Jun. 10” to the medication end date “2017 Jun. 21” of the medication M14. As illustrated in the bar graph in FIG. 2D, the dose of the medication M14 to the patient P1 was small on “2017 Jun. 10” and was subsequently increased gradually until the dose reached a maximum amount on “2017 Jun. 14”. Further, in the time period from “2017 Jun. 15” to “2017 Jun. 17”, the dose was maintained at the maximum amount and was subsequently decreased gradually. The area of the bars in the bar graph in FIG. 2D denotes the total dose “500 mL” in the time period from “2017 Jun. 10” to “2017 Jun. 21”.

Alternatively, as illustrated in FIG. 2D, the display control function 132 may also display, in time series, the doses of the medications M11, M12, M13, M15, M16, M17, M16, and M19. In this situation, the dots (the circular shapes) in FIG. 29 indicate that the medication M11 was administered to the patient P1 one time on “2017 Jun. 10” and three times on “2017 Jun. 11”. Further, the dots in FIG. 2D indicate that the medication M13 and the medication M17 were administered to the patient P1 one time each on “2017 Jun. 10”. Further, the dots in FIG. 2D indicate that the medication M15 was administered to the patient P1 one time on “2017 Jun. 10”, twice on “2017 Jun. 11”, twice on “2017 Jun. 12”, twice on “2017 Jun. 13”, twice on “2017 Jun. 14”, twice on “2017 Jun. 17”, twice on “2017 Jun. 18”, twice on “2017 Jun. 19”, and twice on “2017 Jun. 20”. In this situation, the lines connecting the dots in FIG. 2D indicate the time period from the medication start to the medication end. Alternatively, in FIG. 2D, the display control function 132 does not necessarily have to display the doses of the medications M11, M12, M13, 7115, M16, M17, M18, and M19. Further, when one is selected from among the medications M11, M12, M13, M15, M16, M17, M18, and M19, the display control function 132 may display the doses of the newly-selected medication in time series in a bar graph.

As illustrated in FIG. 2D, after the display control function 132 displays the prescription records in time series, the acquisition function receives an operation performed on the prescription records displayed in time series and acquires a time range. In other words, the acquisition function 133 is configured to receive an operation performed on any of the pieces of second medical data displayed in time series and to acquire the time range corresponding to the received operation.

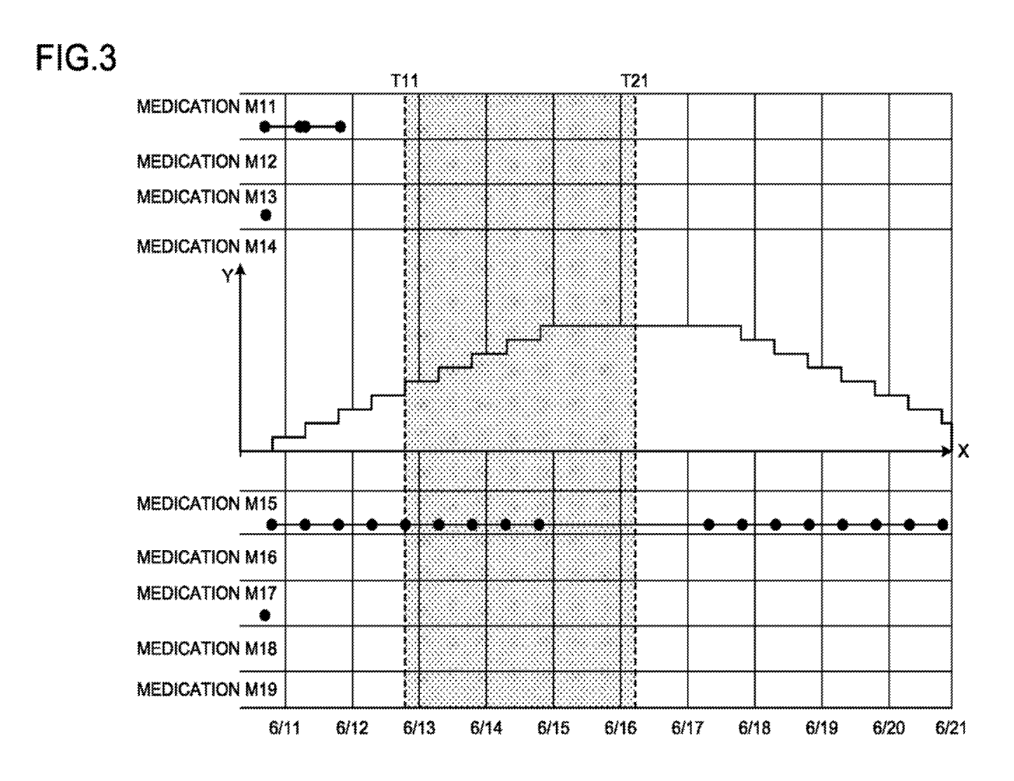

For example, as illustrated in FIG. 3, the acquisition function 133 receives, from the operator, a determination operation to determine a start time T11 and an end time T21 and acquires the time period from the start time T11 to the end time T21 as a time range FIG. 3 is a chart illustrating an example of the time range according to the first embodiment. Further, the start time T11 may be referred to as a first point in time (hereinafter, simply “the first time”). The end time T21 may be referred to as a second point in time (hereinafter, simply “the second time”). In other words, the acquisition function 133 is configured to receive the determination operation to determine the first time and the second time and to acquire the time period from the first time to the second time as the time range.

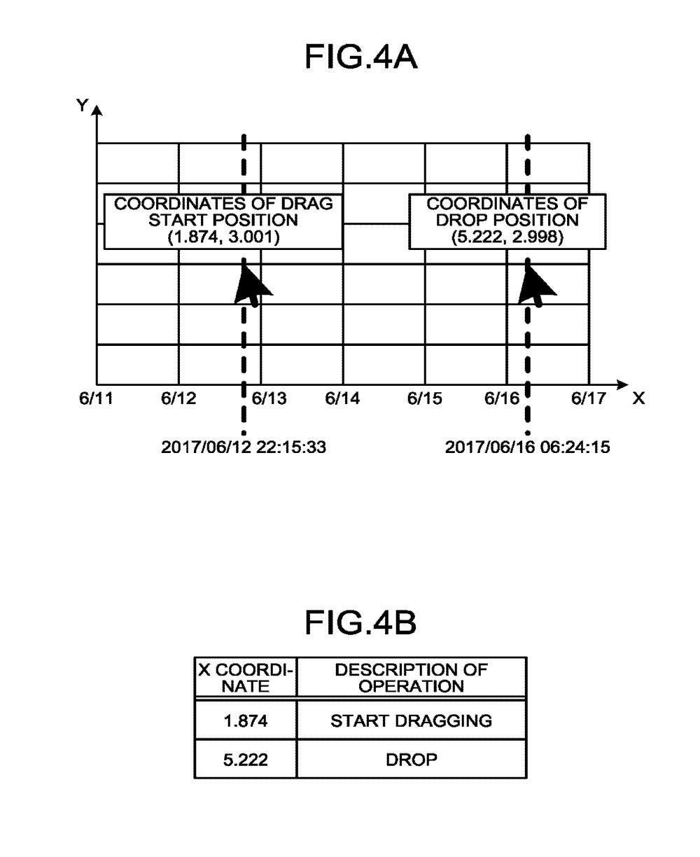

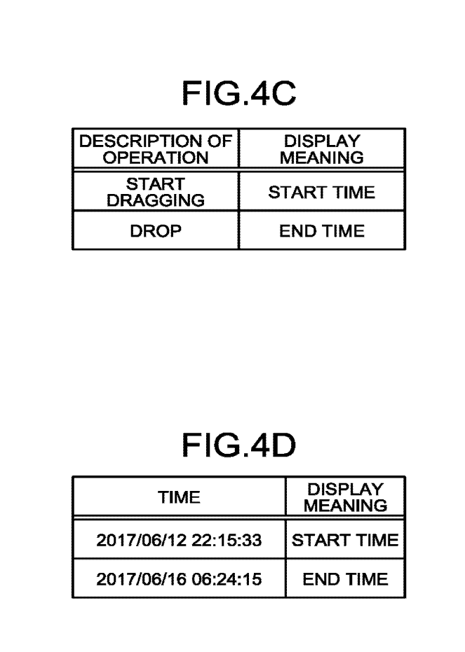

Next, the time range acquiring process will be explained further in detail, with reference to FIGS. 4A, 4E, 4C, and 4D. FIGS. 4A, 4B, 4C, and 4D are drawings for explaining the time range acquiring process according to the first embodiment.

First, by operating the mouse included in the input interface 610, the operator starts a drag operation at the coordinate (1.874,3.001). In this situation, FIG. 4A illustrates icons (arrows) that are manipulatable by the operator via the mouse, on the bar graph illustrated in FIGS. 2D and 3. In this situation, the acquisition function 133 acquires the X coordinate (1.874) extracted from the coordinates (1.874,3.001) of the drag start position and the description of the operation “start dragging”. Subsequently, by operating the mouse included in the input interface 610, the operator performs a drop operation (ends the drag operation) at the coordinates (5.222,2.998). In that situation, the acquisition function 133 acquires the X coordinate (5.222) extracted from the coordinates (5.222,2.998) of the drop position and the description of the operation “drop”. In other words, as illustrated in FIG. 4B, the acquisition function 133 acquires a correspondence relationship between the X coordinates and the descriptions of the operations.

Subsequently, on the basis of the coordinate/time conversion table in FIG. 2B, the acquisition function 133 converts the acquired X coordinates into times. For example, the acquisition function 133 converts the X coordinate (1.874) at which the drag operation was started, into the time “2017 Jun. 12, 22:15:33”. Further, for example, the acquisition function 133 converts the K coordinate (5.222) at which the drop operation was performed, into the time “2017 Jun. 16, 06:24:15”.

Subsequently, on the basis of the correspondence relationship illustrated in FIG. 40, the acquisition function 133 converts the descriptions of the operations into display meanings. In this situation, FIG. 4G illustrates a “description of operation”/“display meaning” conversion table defining the correspondence relationship between descriptions of operations and display meanings. More specifically, the “description of operation”/“display meaning” conversion table in FIG. 40 defines that the description of operation “start dragging” corresponds to a display meaning “start time” and that the description of operation “drop” corresponds to a display meaning “end time”. For example, the acquisition function 133 reads the “description of operation”/“display meaning” conversion table illustrated in FIG. 40 from the memory 120 and further converts the description of operation “start dragging” into the display meaning “start time” and converts the description of operation “drop” into the display meaning “end time”. After that, as illustrated in FIG. 4D, the acquisition function 133 acquires, as a time range, the time “2017 Jun. 12, 22:15:33” indicating the start time T11 and the time “2017 Jun. 16, 06:24:15” indicating the end time T21.

Besides the mouse operation explained above, the acquisition function 133 is capable of acquiring a time range on the basis of other various operations that designate two points in the prescription records displayed in time series. In other words, the acquisition function 133 is capable of acquiring a time range by receiving, from the operator, an operation to designate two points in the prescription records displayed in time series as a determination operation to determine the first time and the second time and further converting each of the X coordinates of the designated two points into a time. In this situation, examples of the operation to designate two points in the prescription records displayed in time series include: a drag & drop operation and a double-click operation using a mouse; and a swipe operation and a double tap operation using a touch panel.

After the acquisition function 133 has acquired the time range, the extracting function 134 extracts medical image data corresponding to the time range from the PACS server 300. In other words, the extracting function 134 is configured to extract at least one of the pieces of first medical data corresponding to the time range, from the PACS server 300. For example, the extracting function 134, at first, extracts pieces of medical image data of the patient. P1 stored in the PACS server 300. In one example, the extracting function 134 extracts the pieces of medical image data of the patient P1, by referring to the DICOM tag “(0010,0020) Patient ID” of the pieces of medical image data stored in the PACS server 300.

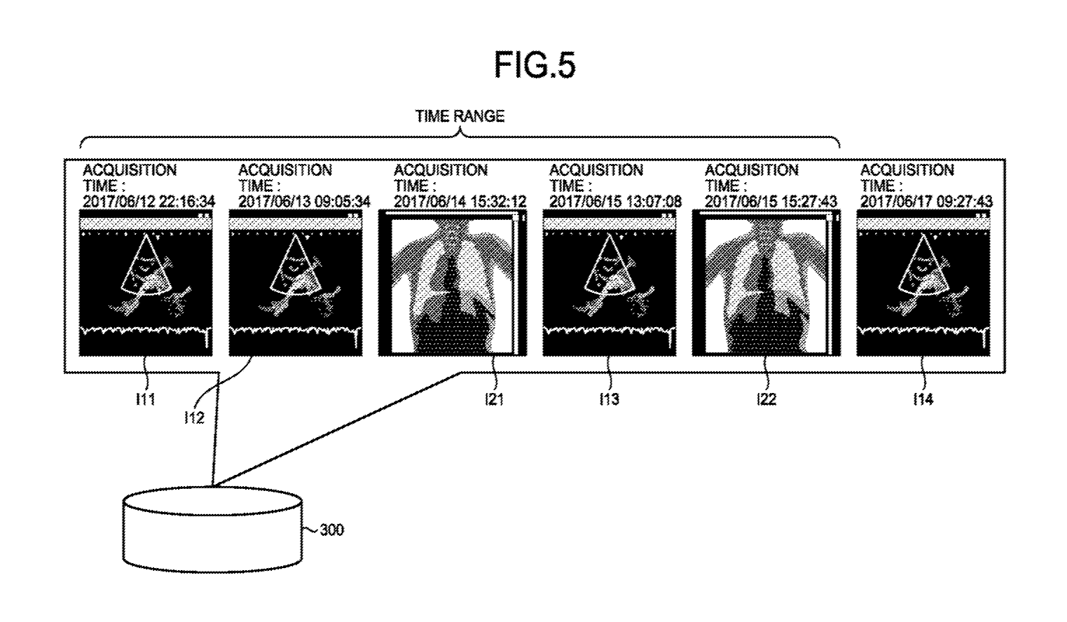

In the following sections, an example will be explained in which, the following are extracted as the pieces of medical image data of the patient P1: Computed Tomography (CT) image data I21 and CT image data I32 (not illustrated); and ultrasonic image data I11, ultrasonic image data I12, X-ray image data I21, ultrasonic image data I13, X-ray image data I22, and ultrasonic image data I14 illustrated in FIG. 5. FIG. 5 is a drawing for explaining the medical image data extracting process according to the first embodiment.

Subsequently, from the pieces of medical image data of the patient P1, the extracting function 134 extracts one or more pieces of medical image data corresponding to the time range. For example, as the pieces of medical image data corresponding to the time range, the extracting function 134 extracts two or more of the pieces of medical image data to be compared with each other that correspond to the time range. In other words, as the pieces of first medical data corresponding to the time range, the extracting function 134 is configured to extract two or more of the pieces of first medical data to be compared with each other that correspond to the time range. In one example, as the two or more of the pieces of medical image data to be compared with each other that correspond to the time range, the extracting function 134 extracts the earliest piece of medical image data and the latest piece of medical image data from among the pieces of medical image data included in the time range. In other words, as the pieces of first medical data to be compared with each other, the extracting function 134 is configured to extract the earliest piece of first medical data and the latest piece of first medical data from among the pieces of first medical data included in the time range. In another example, as the two or more of the pieces of medical image data to be compared with each other that correspond to the time range, the extracting function 134 may extract one of the pieces of medical image data acquired at a time closest to the start time and another one of the pieces of medical image data acquired at a time closest to the end time.

In the following sections, an example will be explained in which, as the two or more of the pieces of medical image data to be compared with each other that correspond to the time range, the extracting function 134 extracts the earliest piece of medical image data and the latest piece of medical image data from among the pieces of medical image data included in the time range. For example, the extracting function 134, at first, acquires the acquisition times of the pieces of medical image data by referring to the DICOM tags “(0008,0022) Acquisition Date” and “(0008,0032) Acquisition Time” and further extract pieces of medical image data included in the time range by comparing the acquisition times with the time range.

In one example, let us discuss a situation in which the acquisition time of the ultrasonic image data I11 is “2017 Jun. 12, 22:16:34”, the acquisition time of the ultrasonic image data I12 is “2017 Jun. 13, 09:05:34”, the acquisition time of the K-ray image data I21 is “2017 Jun. 14, 15:32:12”, the acquisition time of the ultrasonic image data I13 is “2017 Jun. 15, 13:07:08”, the acquisition time of the X-ray image data I22 is “2017 Jun. 15, 15:27:43”, and the acquisition time of the ultrasonic image data I14 is “2017 Jun. 17, 09:27:43”, while the time range is the time period from “2017 Jun. 12, 22:15:33” to “2017 Jun. 16, 06:24:15”. In this situation, the pieces of medical image data included in the time range are, as illustrated in FIG. 5, the ultrasonic image data I11, the ultrasonic image data I12, the X-ray image data I21, the ultrasonic image data I13, and the X-ray image data I22. Accordingly, as the two or more of the pieces of medical image data to be compared with each other that correspond to the time range, the extracting function 134 extracts the ultrasonic image data I11 that is the earliest piece of medical image data and the X-ray image data I22 that is the latest piece of medical image data, from among the pieces of medical image data included in the time range.

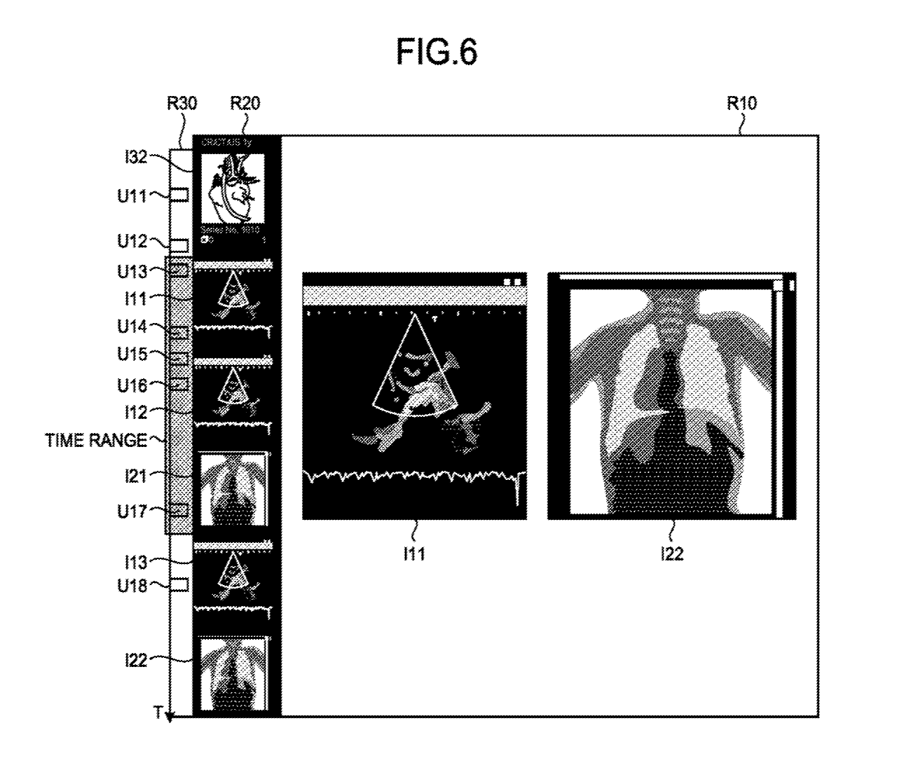

Further, the display control function 132 displays the pieces of medical image data extracted by the extracting function 134. In other words, the display control function 132 is configured to display the pieces of first medical data extracted by the extracting function 134. For example, as illustrated in a region R10 in FIG. 6, the display control function 132 displays the ultrasonic image data I11 and the X-ray image data I22 extracted by the extracting function 134. FIG. 6 is a drawing for explaining the medical image data display process according to the first embodiment. On the display 620, the display control function 132 may present the display in FIG. 6 in together with the display of the prescription records in time series or may present the display in FIG. 6 in place of the display of the prescription records in time series. Further, when the medical information processing system 1 includes a plurality of display devices, the display control function 132 may present the display of the prescription records in time series and the display illustrated in FIG. 6 on mutually-different display devices.

In this situation, as illustrated in a region R20 in FIG. 6, the display control function 132 may display the pieces of medical image data of the patient P1 in thumbnails. For example, the display control function 132 displays, in the thumbnails, the eight pieces of medical image data extracted as pieces of the medical image data of the patient P1 (i.e., the CT image data I31, the CT image data I32, the ultrasonic image data I11, the ultrasonic image data I12, the X-ray image data I21, the ultrasonic image data I13, the X-ray image data I22, and the ultrasonic image data I14).

In that situation, instead of simultaneously displaying the eight pieces of medical image data in the region R20, the display control function 132 may display these pieces of medical image data in a scrollable form. For example, as illustrated in FIG. 6, from among the eight pieces of medical image data, the display control function 132 displays, in the region R20, six pieces of medical image data, namely the CT image data I32, the ultrasonic image data ill, the ultrasonic image data I12, the X-ray image data I21, the ultrasonic image data I13, and the X-ray image data I22 in thumbnails. After that, in accordance with a mouse wheel operation or a touch panel swipe operation performed by the operator via the input interface 610, the display control function 132 scrolls the images displayed in the thumbnails.

Further, as illustrated in a region R30 in FIG. 6, the display control function 132 may display a bar indicating the times corresponding to the pieces of medical image data displayed in the thumbnails. The bar in the region R30 in FIG. 6 indicates the acquisition times of the pieces of medical image data on a time axis T of which the bottom direction corresponds to the positive direction. In other words, the display control function 132 is configured to display the pieces of first medical data stored in the PACS server 300 in the thumbnails and to also display the bar indicating the times corresponding to the pieces of first medical data.

More specifically, the display control function 132, at first, adjusts a time scale on the basis of the earliest acquisition time and the most recent acquisition time among the acquisition times of the pieces of medical image data displayed in the thumbnails. Further, as illustrated in the region R30 in FIG. 6, the display control function 132 displays, on the tune axis T of which the time scale has been adjusted, an acquisition time U11 of the CT image data I31, an acquisition time U12 of the CT image data I32, an acquisition time U13 of the ultrasonic image data I11, an acquisition time U14 of the ultrasonic image data I12, an acquisition time U15 of the X-ray image data I21, an acquisition time U16 of the ultrasonic image data I13, an acquisition time U17 of the X-ray image data I22, and an acquisition time U18 of the ultrasonic image data I14, so as to be associated with the time axis T.

In this situation, as illustrated in FIG. 6, the display control function 132 may display the time range in the bar in the region R30. When the time range is displayed, the operator is able to easily determine during which time period, the pieces of medical image data displayed in the region R10 were acquired. For example, in the example in FIG. 6, the operator is able to intuitively understand that the ultrasonic image data I11 displayed in the region R10 was acquired at the acquisition time P13 soon after the treatment for the patient P1 was started and that the X-ray image data I22 was acquired at the acquisition time U17 that was in later time period of the treatment for the patient P1.

With reference to FIG. 6, the example was explained in which the time range is displayed in the bar 30 within the region R30; however, possible embodiments are not limited to this example. For instance, the display control function 132 may display the time range by using numerical values or text.

In one example, as illustrated in FIG. 4D, the display control function 132 may display the time range in a table indicating the start time and the end time.

With reference to FIGS. 5 and 6, the example was explained in which, as the pieces of medical image data corresponding to the time range, the earliest piece of medical image data and the latest piece of medical image data are extracted from among the pieces of medical image data included in the time range. Alternatively, the extracting function 134 may be configured to extract pieces of medical image data corresponding to the time range, while taking the types of the medical image data into consideration.

For example, the PACS server 300 stores therein a plurality of types of medical image data. In other words, the PACS server 300 is configured to store therein the pieces of first medical data of the plurality of types. Further, the extracting function 134 is configured to receive, from the operator, a selecting operation to select one of the types of which medical image data is to be extracted. In this situation, for example, the types of the medical image data may be the types of modality (e.g., ultrasonic diagnosis apparatuses, X-ray diagnosis apparatuses, X-ray CT apparatuses, Magnetic Resonance Imaging (MRI) apparatuses, Single Photon Emission Computed Tomography (SPECT) apparatuses, Positron Emission computed Tomography (PET) apparatuses). The number of types of medical image data selected by the selecting operation may be two or more.

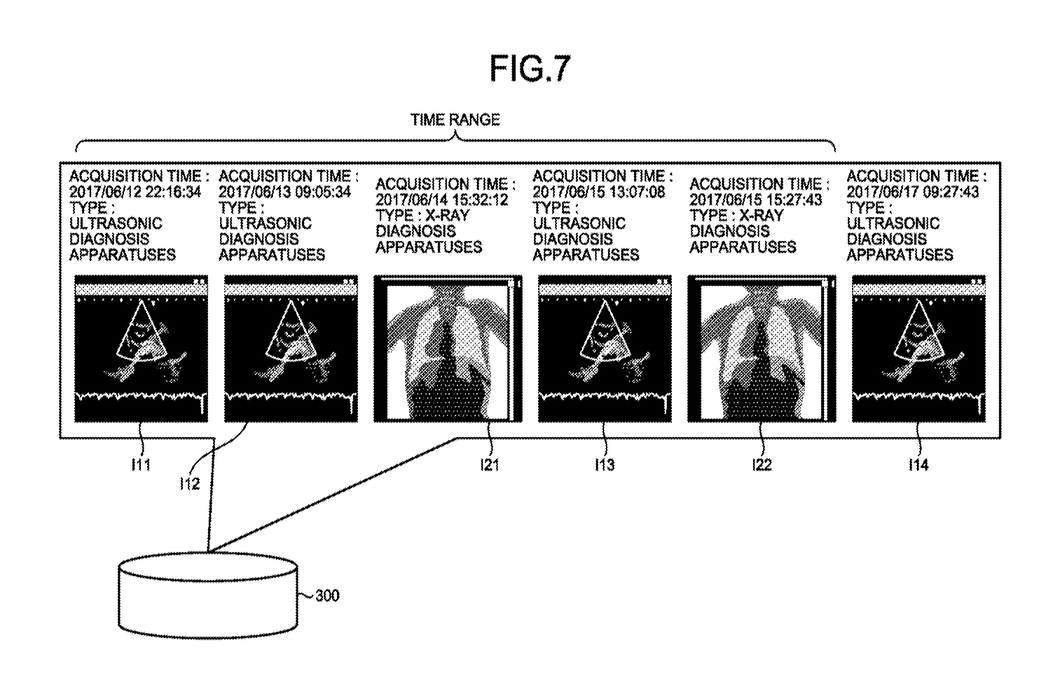

In one example, the extracting function 134 receives a selecting operation to select “ultrasonic diagnosis apparatuses” and “X-ray diagnosis apparatuses” as the types of the medical image data. Further, as illustrated in FIG. 7, from among the pieces of medical image data of the patient P1, the extracting function 134 extracts one or more pieces f medical image data that are of the selected types and are included in the time range. In other words, the extracting function 134 extracts the ultrasonic image data I11, the ultrasonic image data I12, the X-ray image data I21, the ultrasonic image data I13, and the X-ray image data I22, which are pieces of medical image data corresponding to “ultrasonic diagnosis apparatuses” and “X-ray diagnosis apparatuses” and being included in the time range. FIG. 7 is another drawing for explaining the medical image data extracting process according to the first embodiment.

In this situation, for example, the extracting function 134 extracts, as the pieces of medical image data corresponding to the time range, two or more of the pieces of medical image data to be compared with each other that are of the selected types and correspond to the time range. In other words, the extracting function 134 is configured to extract, as the pieces of first medical data corresponding to the time range, the two or more of the pieces of first medical data to be compared with each other that are of the selected types and correspond to the time range. For example, from among the pieces of medical image data corresponding to “ultrasonic diagnosis apparatuses” and “X-ray diagnosis apparatuses” and being included in the time range, the extracting function 134 extracts the ultrasonic image data I11, which is the earliest piece of medical image data, and the X-ray image data I22, which is the latest piece of medical image data.

In another example, as the pieces of medical image data corresponding to the time range, the extracting function 134 extracts, from among the pieces of medical image data that are of the selected types and are included in the time range, two or more of the pieces of medical image data to be compared with each other that correspond to the time range for each of the types of medical image data. In other words, as the pieces of first medical data corresponding to the time range, the extracting function 134 is configured to extract, for each of the types of first medical data, two or more of the pieces of first medical data to be compared with each other that correspond to the time range. For example, from among the pieces of medical image data corresponding to “ultrasonic diagnosis apparatuses” and being included in the time range, the extracting function 134 extracts the ultrasonic image data I11, which is the earliest piece of medical image data, and the ultrasonic image data I13, which is the latest piece of medical image data. Further, from among the pieces of medical image data corresponding to “X-ray diagnosis apparatuses” and being included in the time range, the extracting function 134 extracts the X-ray image data I21, which is the earliest piece of medical image data, and the X-ray image data I22, which is the latest piece of medical image data.

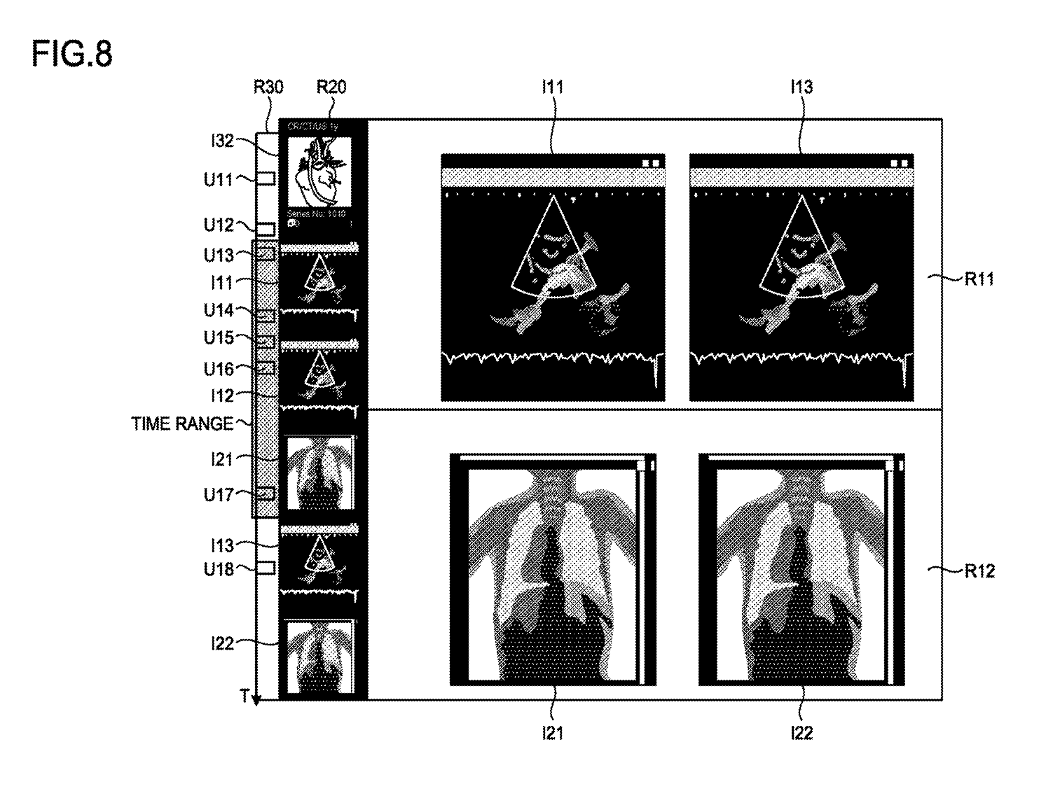

In that situation, as illustrated in a region R11 in FIG. 8, the display control function 132 displays the ultrasonic image data I11 and the ultrasonic image data I13 extracted by the extracting function 134. Further, as illustrated in a region R12 in FIG. 8, the display control function 132 displays the X-ray image data I21 and the X-ray image data I22 extracted by the extracting function 134. FIG. 8 is another drawing for explaining the medical image data display process according to the first embodiment.

In this situation, as illustrated in the region R20 in FIG. 8, the display control function 132 may display the pieces of medical image data of the patient P1 in thumbnails. Further, as illustrated in the region R30 in FIG. 8, the display control function 132 may display a bar indicating the times corresponding to the pieces of medical image data displayed in the thumbnails. Further, as illustrated in FIG. 8, the display control function 132 may be configured to display the time range in the bar within the region R30.

The example is explained above in which the types of the medical image data are classified by the modalities; however, possible embodiments are not limited to this example. For instance, pieces of medical image data that were acquired by using the same type of modality but were acquired in mutually-different acquisition modes may be considered as pieces of medical image data of mutually-different types. In one example, a piece of B-mode image data and a piece of Doppler image data that are both acquired by using an ultrasonic diagnosis apparatus may be considered as pieces of medical image data of mutually-different types.

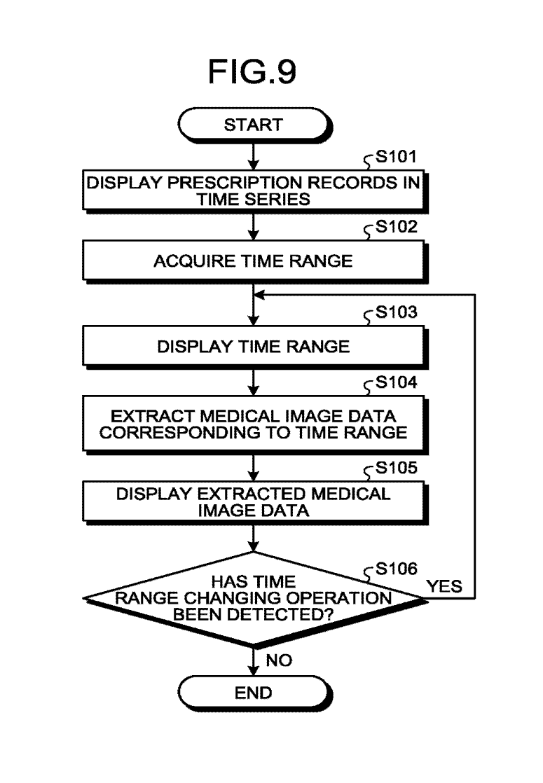

Next, an example of a procedure of processes performed by the medical information processing system 1 will be explained, with reference to FIG. 9 FIG. 9 is a flowchart for explaining a flow in a series processes performed by the medical information processing system 1 according to the first embodiment. Steps S101, S103, and S105 are steps corresponding to the display control function 132. Steps S102 and S106 are steps corresponding to the acquisition function 133. Step S104 is a step corresponding to the extracting function 134.

First, the processing circuitry 130 reads the prescription records stored in the electronic medical record storage apparatus 400 while being associated with the times and displays the prescription records in time series on the display 620 (step S101). Subsequently, the processing circuitry 130 receives an operation performed on the prescription records displayed in time series, acquires a time range corresponding to the received operation (step S102), and displays the acquired time range (step S103).

After that, the processing circuitry 130 extracts the pieces of medical image data corresponding to the acquired time range from the PACS server 300 (step S104) and displays the extracted pieces of medical image data on the display 620 (step S105). In this situation, the processing circuitry 130 judges whether or not an operation to change the time range has been detected (step 106). When an operation to change the time range has been detected, the processing circuitry 130 returns to step S103 (step S106: Yes). On the contrary, when no operation to change the time range has been detected (step S106: No), the processing circuitry 130 ends the process.

The order in which step S103 and steps S104 and S105 are performed is arbitrary. For example, the processing circuitry 130 may extract the pieces of medical image data corresponding to the time range, display the extracted pieces of medical image data, and subsequently display the time range. In another example, the processing circuitry 130 may extract the pieces of medical image data corresponding to the time range, display the time range, and subsequently display the extracted pieces of medical image data. Further, the processing circuitry 130 does not necessarily have to perform the process at step S103.

As explained above, according to the first embodiment, the PACS server 300, the electronic medical record storage apparatus 400, and the specimen examination server 500 are each configured to store therein the medical image data and the prescription records so as to be associated with the times. The acquisition function 133 is configured to receive the operation performed on the prescription records displayed in time series and to acquire the time range corresponding to the received operation. The extracting function 134 is configured to extract the pieces of medical image data corresponding to the time range from the PACS server 300. The display control function 132 is configured to display the pieces of medical image data extracted by the extracting function 134.

With these arrangements, for example, by simply designating a medication administration time period, the operator who has referred to the prescription records displayed in time series is able to view the piece of medical image data corresponding to the time when the administration of the medication was started and the piece of medical image data corresponding to either the time when the administration of the medication was ended or the current point in time. In other words, the medical information processing system 1 according to the first embodiment is able to make it easy to compare the prescription records with the medical image data and to further make it easy to adjust the types and the doses of the medications.

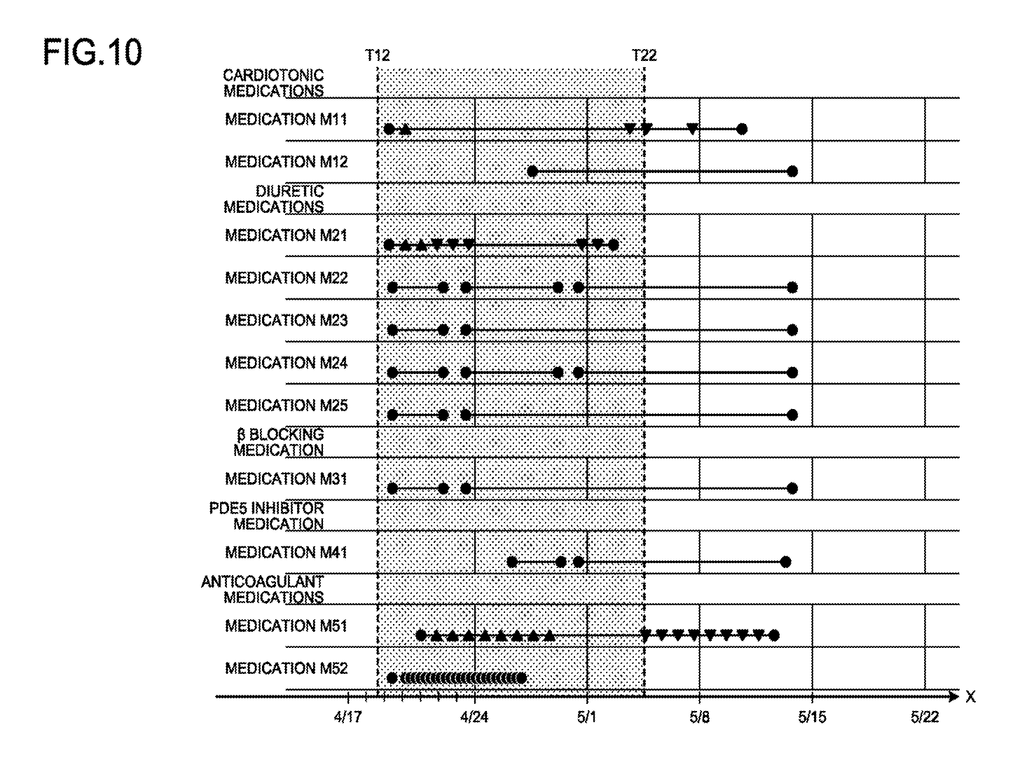

As the display of the prescription records in time series, only the one example in FIG. 2D has so far been explained; however, possible embodiments are not limited to this example. Next, another example of displaying prescription records in time series will be explained, with reference to FIG. 10. FIG. 10 is a drawing illustrating the example of displaying prescription records in time series according to the first embodiment.

For example, at first, the display control function 132 receives, from the operator, a selecting operation to select a patient, via the input interface 610. In the following sections, an example will be explained in which a patient 64 is selected as the patient. Subsequently, the display control function 132 displays the doses of the medications to the patient P4 in time series. More specifically, as illustrated in FIG. 10, the display control function 132 displays, in time series, the doses to the patient P4 with regard to the following medications: medications M11 and M12 that are each a cardiotonic medication; medications M21, M22, M23, M24, and M25 that are each a diuretic medication; a medication M31 that is β blocking medication; a medication M41 that is a PDE5 inhibitor medication; and medications M51 and M52 that are each an anticoagulant medication.

Even more specifically, in FIG. 10, the display control function 132 indicates the medication start dates and the medication end dates, by using dot shapes and straight lines each connecting two or more of the shapes. For example, in FIG. 10, the display control function 132 indicates that, to the patient P4, the medication M11 was administered every day in the time period from “2017 Apr. 19” to “2017 May 10” and that the medication M12 was administered every day in the time period from “2017 Apr. 27” to “2017 May 14”.

Further, in FIG. 10, the display control function 132 displays transitions in the doses of the medications by using triangular shapes and inverted triangular shapes. For example, in FIG. 10, the display control function 132 indicates that the medication M11 was administered by a basic dose “15 mg” on “20 Apr. 19”, that the dose was subsequently increased by a unit weight of “5 mg” on “2017 Apr. 20”, and that the medication M11 was administered in dosage of “20 mg” from “2017 Apr. 20” to “2017 May 2”. Further, in FIG. 10, the display control function 132 indicates that the dose was decreased by the unit weight of “5 mg” on “2017 May 3” and that the medication M11 was administered in dosage of “15 mg” on “2017 May 3”. Further, in FIG. 10, the display control function 132 indicates that the dose was decreased by the unit weight of “5 mg” on “2017 May 4” and that the medication M11 was administered in dosage of “10 mg” from “2017 May 4” to “2017 May 6”. Further, in FIG. 10, the display control function 132 indicates that the dose was decreased by the unit weight of “5 mg” on “2017 May 7”, that the medication M11 was administered in dosage of “5 mg” from “2017 May 7” to “2017 May 10”, and that the administration of the medication M11 was ended on “2017 May 10”.

After the display control function 132 has the prescription records displayed in time series as illustrated in FIG. 10, the acquisition function 133 receives an operation performed on the prescription records displayed in time series and acquires a time range. For example, the acquisition function 133 acquires a start time T12 and an end time T22 as the time range, by receiving, from the operator, an operation to designate two points in FIG. 10. After that, the extracting function 134 extracts pieces of medical image data corresponding to the time range from the PACS server 300. The display control function 132 displays the extracted pieces of medical image data.

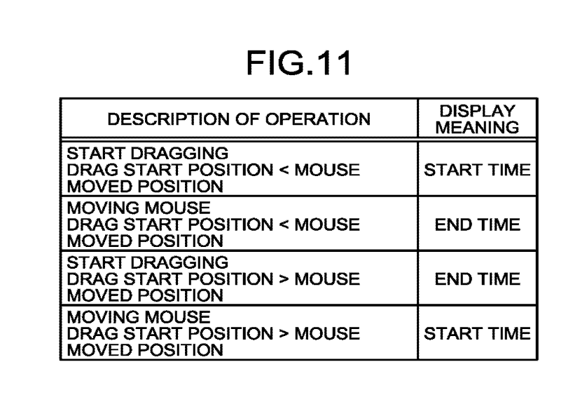

Further, the example is explained above in which the time range is acquired as a result of the determination operation to determine the first time and the second time; however, possible embodiments are not limited to this example. For instance, the acquisition function 133 may receive a determination operation to determine the first time and may successively acquire time periods each from the first time to a second time that is successively changeable, as time ranges. In the following sections, this feature will be explained with reference to FIG. 11. FIG. 11 is yet another table for explaining the time range acquiring process according to the first embodiment.

First, by operating the mouse included in the input interface 610, the operator moves the icon by starting a drag operation in an arbitrary position in the prescription records displayed in time series. In s situation, the acquisition function 133 acquires the X coordinate of the drag start position and the description of the operation “start dragging”. Further, the acquisition function 133 successively acquires the X coordinates of the mouse moved positions (the current positions of the icon).

Subsequently, on the basis of the coordinate/time conversion table in FIG. 2B, the acquisition function 133 converts the acquired X coordinates into times. Further, on the basis of the correspondence relationship illustrated in FIG. 11, the acquisition function 133 converts the description of the operation into a display meaning. In this situation, FIG. 11 illustrates a “description of operation”/“display meaning” conversion table defining a correspondence relationship between descriptions of operations and display meanings. More specifically, the “description operation” “display meaning” conversion table illustrated in FIG. 11 indicates that when “drag start position<mouse moved position” is satisfied (i.e., when the X coordinate of the drag start position is smaller than the X coordinate of the current position of the icon), the description of operation “start dragging” corresponds to display meaning “start time”, whereas the description of operation “moving the mouse” corresponds to a display meaning “end time”. Further, the “description of operation”/“display meaning” conversion table illustrated in FIG. 11 indicates that when “drag start position>mouse moved position” is satisfied (i.e., when the X coordinate of the drag start position is larger than the X coordinate of the current position of the icon), the description of operation “start dragging” corresponds to a display meaning “end time”, whereas the description of operation “moving the mouse” corresponds to a display meaning “star time”. Further, by converting the descriptions of the operations into the display meanings according to the “description of operation”/“display meaning” conversion table, the acquisition function 133 acquires the start time and the end time so as to acquire the time range.

In this situation, the time acquired by converting the X coordinate of the drag start position will be referred to as the first time, whereas the time acquired by converting the X coordinate of the mouse moved position will be referred to as the second time. In other words, when “drag start position mouse moved position” is satisfied, the start time will be referred to as the first time, whereas the end time will be referred to as the second time. On the contrary, when “drag start position>mouse moved position” is satisfied, the end time will be referred to as the first time, whereas the start time will be referred to as the second time.

As explained above, as a result of the dragging operation being started, the acquisition function 133 receives the determination operation to ermine the first time. Further, the acquisition function 133 successively acquires the time periods each from the first time to the second time that is successively changeable in accordance with the mouse operation, as the time ranges. In this situation, every time the acquisition function 133 newly acquires a time range, the extracting function 134 extracts pieces of medical image data corresponding to the new time range. In other words, every time the acquisition function 133 newly acquires a time range, the extracting function 134 is configured to extract pieces of first medical data corresponding to the newly-acquired time range. Further, every time the extracting function 134 extracts pieces of medical image data, the display control function 132 displays the newly-extracted pieces of medical image data on the display 620. In other words, every time the extracting function 134 extracts pieces of first medical data, the display control function 132 is configured to display the newly-extracted pieces of first medical data. With these arrangements, the display 620 dynamically displays the pieces of medical image data in conjunction with the operations performed by the operator who viewed the prescription records displayed in time series.

In the first embodiment described above, the example is explained in which the operation performed on the pieces of non-image data displayed in time series is received, so as to acquire the time range corresponding to the received operation. In contrast, in a second embodiment, an example will be explained in which an operation performed on pieces of image data displayed in time series is received so as to acquire a time range corresponding to the received operation.

The medical information processing system 1 according to the second embodiment has a configuration similar to the configuration of the medical information processing system 1 illustrated in FIG. 1, except that a part of the processes performed by the display control function 132 and the acquisition function 133 is different. Thus, some of the constituent elements that are the same as those explained in the first embodiment will be referred to by using the same reference characters as in FIG. 1, and the explanations thereof will be omitted.

First, the display control function 132 displays pieces of first medical data in time series. For example, as the pieces of first medical data, the display control function 132 displays, in tune series, at least one selected from between pieces of medical image data and reports. In the following sections, an example will be explained in which pieces of medical image data are displayed in time series, as the pieces of first medical data.

For example, at first, the display control function 132 receives, from the operator, a selecting operation to select a patient, via the input interface 610. Subsequently, the display control function 132 extracts pieces of medical image data of the selected patient (hereinafter, “the patient P1”). For example, by referring to the DICOM tag “(0010,0020) Patient ID” of the pieces of medical image data stored in the PACS server 300, the display control function 132 extracts, as the pieces of medical image data of the patient P1, the CT image data I31, the CT image data I32, the ultrasonic image data I11, the ultrasonic image data I12, the X-ray image data I21, the ultrasonic image data I13, the X-ray image data I22, and the ultrasonic image data I14.

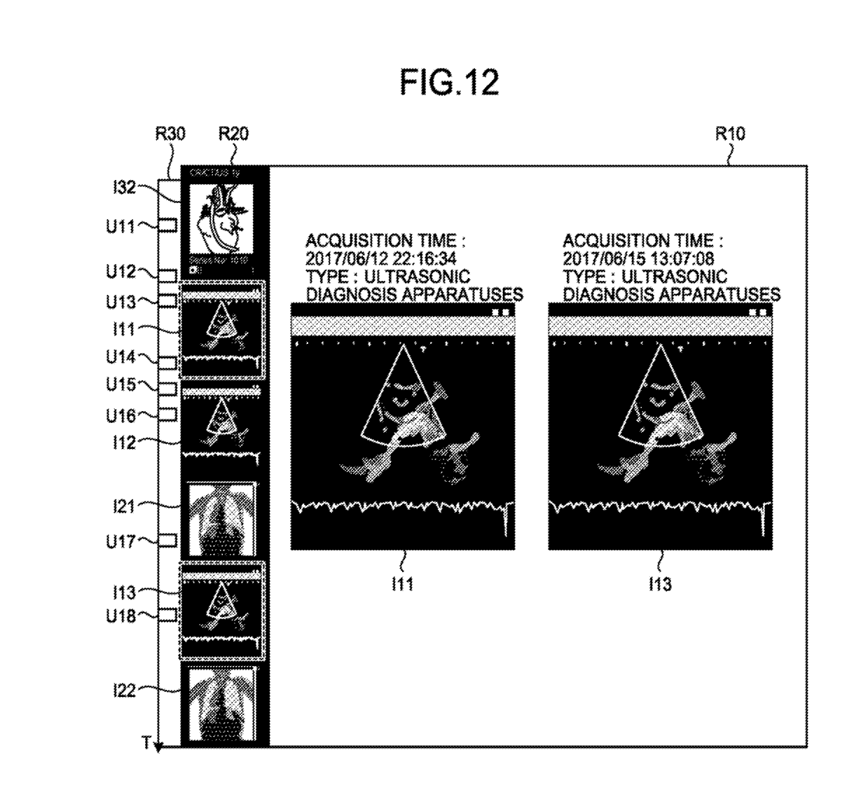

After that, the display control function 132 displays the pieces of medical image data of the patient P1 in time series. For example, as illustrated in the region R20 in FIG. 12, the display control function 132 displays the pieces of medical image data of the patient P1 in time series. FIG. 12 is a drawing for explaining the displaying of the pieces of medical image data in time series according to the second embodiment.

More specifically, in the region R20 in FIG. 12, the display control function 132 displays, in thumbnails, the eight pieces of medical image data extracted as the pieces of medical image data of the patient P1 (namely, the CT image data I31, the CT image data I32, the ultrasonic image data I11, the ultrasonic image data I12, the X-ray image data I21, the ultrasonic image data I13, the X-ray image data I22, and the ultrasonic image data I14). In this situation, instead of simultaneously displaying the eight pieces of medical image data in the region R20, the display control function 132 may display these pieces of medical image data in a scrollable form.

Further, as illustrated in the region R30 in FIG. 12, the display control function 132 displays a bar indicating the times corresponding to the pieces of medical image data displayed in the thumbnails. The bar in the region R30 in FIG. 12 indicates the acquisition times of the pieces of medical image data on a time axis T of which the bottom direction corresponds to the positive direction. More specifically, the bar in the region R30 in FIG. 12 displays, on the time axis T, the acquisition time U11 of the CT image data I31, the acquisition time U12 of the CT image data I32, the acquisition time U13 of the ultrasonic image data I11, the acquisition time U14 of the ultrasonic image data I12, the acquisition time U15 of the X-ray image data I21, the acquisition time U16 of the ultrasonic image data I13, the acquisition time U17 of the X-ray image data I22, and the acquisition time U18 of the ultrasonic image data I14 so as to be associated with the time axis T.

In this situation, the acquisition function 133 receives an operation performed on the pieces of medical image data displayed in time series and acquires the time range corresponding to the operation. In other words, the acquisition function 133 is configured to receive the operation performed on the pieces of first medical data displayed in time series and to acquire the time range corresponding to the received operation.

For example, in the region R20, the acquisition function 133 receives a selecting operation to select a first piece of image data and a second piece of image data. In one example, as a result of a mouse operation via the input interface 610, the acquisition function 133 receives, from the operator, a selecting operation to select the ultrasonic image data I11 and the ultrasonic image data I13. Further, the acquisition function 133 acquires the times corresponding to the ultrasonic image data I11 and the ultrasonic image data I13 that were selected. For example, by referring to the DICOM tags “(0008,0022) Acquisition Date” and “(0008,0032) Acquisition Time”, the extracting function 134 acquires the acquisition time “2017 Jun. 12, 22:16:34” as the time corresponding to the ultrasonic image data I11 and the acquisition time “2017 Jun. 15, 13:07:08” as the time corresponding to the ultrasonic image data I13.

In this situation, the display control function 132 may further be configured to display the first piece of image data and the second piece of image data that were selected from the thumbnail display in the region R20, in an enlarged manner. For example, in the region R10 in FIG. 12, the display control function 132 displays the ultrasonic image data I11 and the ultrasonic image data I11 that were selected. Further, as illustrated in the region R10 in FIG. 12, the display control function 132 may be configured to display the acquisition times and the types of the first piece of image data and the second piece of image data. Further, the acquisition function 133 may be configured to receive an operation to change the first piece of image data and the second piece of image data, from the operator who viewed the first piece of image data and the second piece of image data displayed in the region R10.

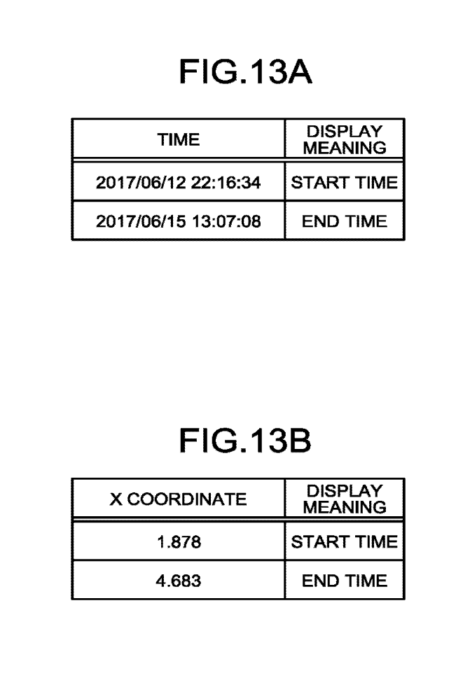

Subsequently, on the basis of the first piece of image data and the second piece of image data, the acquisition function 133 acquires a time range. For example, at first, the acquisition function 133 acquires display meanings with respect to the time corresponding to the first piece of image data and the time corresponding to the second piece of image data. In one example, when the acquisition time of the ultrasonic image data T11 is “2017 Jun. 12, 22:16:34”, whereas the acquisition time of the ultrasonic image data I13 is “2017 Jun. 15, 13:07:08”, the acquisition function 133 acquires, as illustrated in FIG. 13A, a “start time” as the display meaning of “2017 Jun. 12, 22:16:34”, which is the earlier time and further acquires an “end time” as the display meaning of “2017 Jun. 15, 13:07:08”, which is the later time. In this situation, the ultrasonic image data I11, which was acquired at the earlier time, will be referred to as the first piece of image data, whereas the ultrasonic image data I13, which was acquired at the later time, will be referred to as the second piece of image data. In other words, from among the pieces of first medical data displayed in time series, the acquisition function 133 is configured to receive the selecting operation to select the first piece of image data and the second piece of image data and to further acquire the time period from the time corresponding to the first piece of image data to the time corresponding to the second piece of image data as the time range. FIG. 13A is a table for explaining the time range acquiring process according to the second embodiment.

Further, the acquisition function 133 converts the time corresponding to the first piece of image data and the time corresponding to the second piece of image data each into an X coordinate. For example, on the basis of the coordinate/time conversion table in FIG. 2B, the acquisition function 133 converts the acquisition time “2017 Jun. 12, 22:16:34” of the ultrasonic image data I11 into the X coordinate (1.878) and further converts the acquisition time “2017 Jun. 15, 13:07:08” of the ultrasonic image data I13 into the X coordinate (4.683). Accordingly, the acquisition function 133 acquires the correspondence relationship between the X coordinates and the display meanings as illustrated in FIG. 13B. FIG. 13B is a table for explaining the time range display process according to the second embodiment.

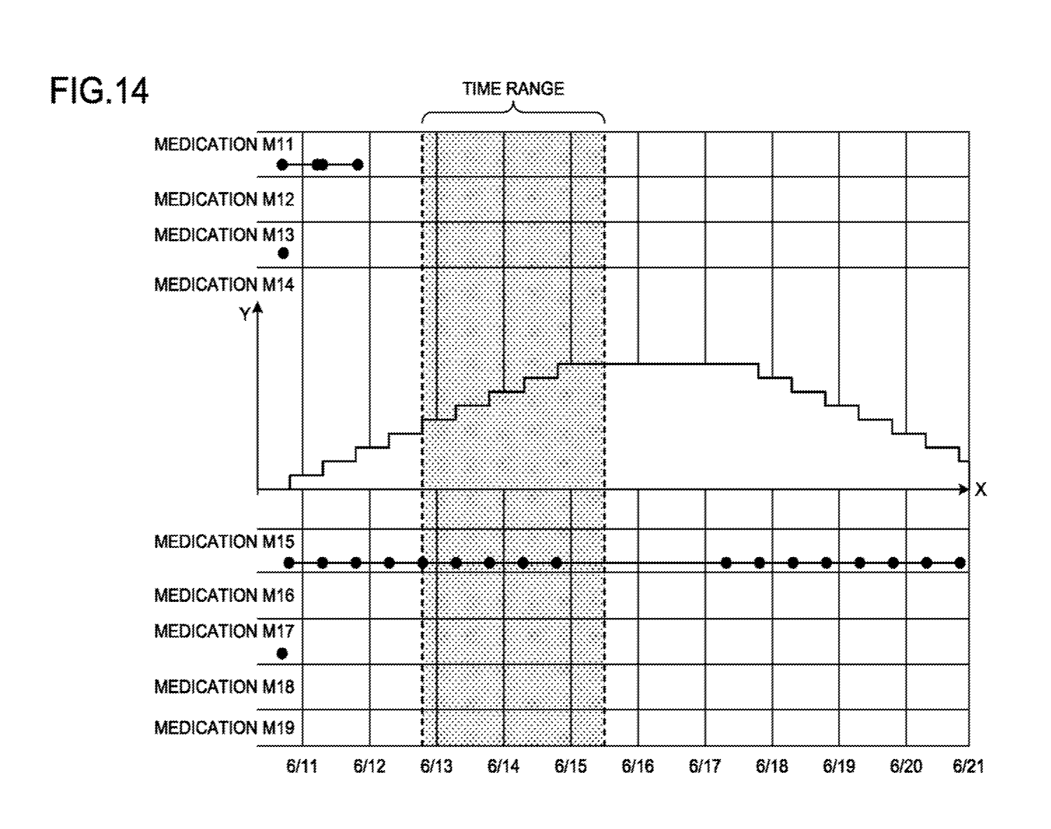

After that, the display control function 132 displays the prescription records and the time range in time series so as to be associated each other. In other words, the display control function 132 is configured to display the pieces of second medical data and the time range in time series, so as to be associated each other. For example, at first, the display control function 132 displays the doses of the selected medication M14 to the selected patient P1, in time series. In one example, the display control function 132 displays the doses in time series by using a bar graph as illustrated in FIG. 14, by plotting the administered times and the doses of the medication M14 to the patient P1 according to the X coordinates illustrated in FIG. 28 and the Y coordinates illustrated in FIG. 20. Further, for example, with respect to the administration of the medications M11, M12, M13, M12, M16, M17, M18, and M19 to the patient P1, the display control function 132 displays the doses in time series by plotting the points in time of the medication administration. After that, as illustrated in FIG. 14, the display control function 132 displays the time range so as to be associated with the doses displayed in time series, by displaying the time range so as to be superimposed on the display of the doses in time series. In other words, the display control function 132 is configured to display, in time series, the doses of the selected medication to the selected patient as the prescription records and to further display the time range so as to be associated with the doses displayed in time series. FIG. 14 is a drawing for explaining the time range display process according to the second embodiment.

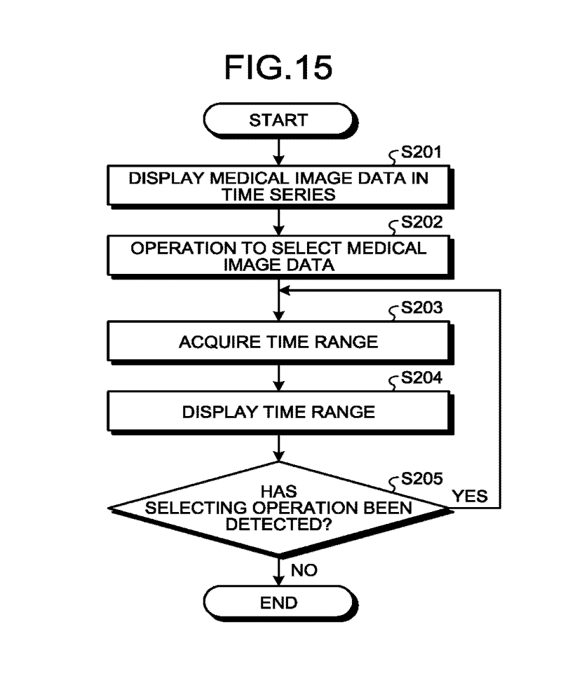

Next, an example of a procedure of processes performed by the medical information processing system 1 will be explained, with reference to FIG. 15. FIG. 15 is a flowchart for explaining a flow in a series of processes performed by the medical information processing system 1 according to the second embodiment. Steps S201 and S204 are steps corresponding to the display control function 132. Steps S202, S203, and S205 are steps corresponding to the acquisition function 133.

First, the processing circuitry 130 reads the pieces of medical image data stored in the FAGS server 300 while being associated with times and displays the read pieces of medical image data in time series on the display 620 (step S201). Subsequently, the processing circuitry 130 receives a selecting operation to select a first piece of image data and a second piece of image data from among the pieces of medical image data displayed in time series (step S202) and acquires a time range corresponding to the received selecting operation (step S203).

Subsequently, the processing circuitry 130 displays, in time series, the acquired time range so as to be associated with prescription records (step S204). In this situation, the processing circuitry 130 judges whether or not a selecting operation to select a first piece of image data and a second piece of image data has been detected again (step S205). When the selecting operation has been detected again, the processing circuitry 130 returns to step S203 (step S205: Yes). On the contrary, when the selecting operation has not been detected again (step S205: No), the processing circuitry 130 ends the process.

As explained above, according to the second embodiment, the FAGS server 300, the electronic medical record storage apparatus 400, and the specimen examination server 500 are each configured to store therein the medical image data and the prescription records so as to be associated with the times. The acquisition function 133 is configured to receive the operation performed on the pieces of medical image data displayed in time series and to acquire the time range corresponding to the received operation. The display control function 132 is configured to display, in time series, the prescription records and tree time range so as to be associated each other.

With these arrangements, for example, by simply selecting a piece of medical image data, the operator is able to check to see how medications were administered before and after the acquisition time of the piece of medical image data. In other words, the medical information processing system 1 according to the second embodiment makes it easy to compare the medical image data with the prescription records and to further make it easy to adjust the types and the doses of the medications.

In the first and the second embodiments described above, the examples are explained in which the medical image data is used as an example of the image data. In contrast, in a third embodiment, variations of the image data will be explained. The medical information processing system 1 according to the third embodiment has a configuration similar to the configuration of the medical information processing system 1 illustrated in FIG. 1, except that a part of the processes performed by the display control function 132 and the extracting function 134 is different. Thus, some of the constituent elements that are the same as those explained in the first embodiment will be referred to by using the same reference characters as in FIG. 1, and the explanations thereof will be omitted.

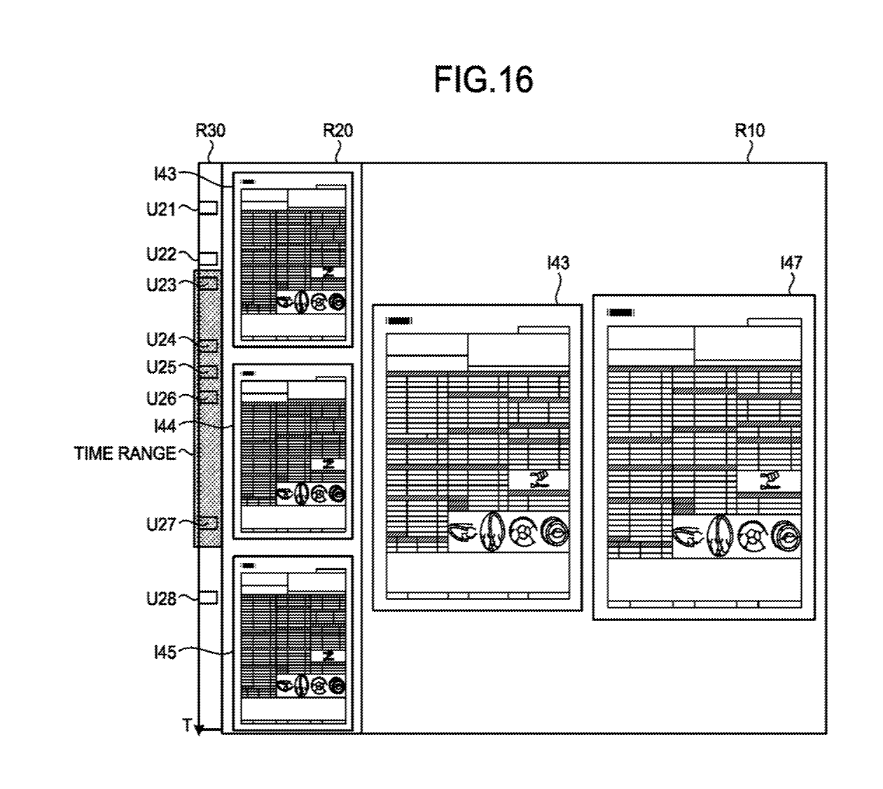

First, an example in which the extracting function 134 extracts reports as pieces of image data corresponding to a time range will be explained, with reference to FIG. 16. FIG. 16 is a drawing for explaining a report display process according to the third embodiment.

For example, the display control function 132, at first, displays the prescription records of a selected patient (hereinafter, “the patient P1”) in time series. Subsequently, the acquisition function 133 receives an operation performed on the prescription records displayed in time series and acquires a time range corresponding to the received operation. After that, the extracting function 134 extracts reports of the patient P1, from among the reports stored in the PACS server 300 while being associated with times at which the reports were created and patient IDs. In the following sections, an example will be explained in which, as the reports of the patient P1, reports 141, 142, 143, 144, 145, 146, 147, and 148 have been extracted.

Subsequently, from among the reports of the patient P1, the extracting function 134 extracts reports corresponding to the time range. For example, by comparing the report creation times of the reports with the time range, the extracting function 134 extracts, as the reports corresponding to the time range, the earliest report and the latest report included in the time range. In the following sections, an example will be explained in which the earliest report included in the time range is the report 143, whereas the latest report included in the time range is the report 147. In this situation, as illustrated in the region R10 in FIG. 16, the display control function 132 displays the report 143 and the report 147 extracted by the extracting function 134.

In this situation, as illustrated in the region R20 in FIG. 6, the display control function 132 may display the reports of the patient P1 in thumbnails. For example, the display control function 132 displays the eight reports extracted as the reports of the patient P1 (namely, the reports 141, 142, 143, 144, 145, 146, 147, and 148) in the thumbnails. In that situation, instead of simultaneously displaying the eight reports in the region R20, the display control function 132 may display these reports in a scrollable form.

Further, as illustrated in the region R30 in FIG. 16, the display control function 132 may display a bar indicating the times corresponding to the reports displayed in the thumbnails. The bar in the region R30 in FIG. 16 indicates the creation times of the reports on a time axis T of which the bottom direction corresponds to the positive direction. More specifically, the bar in the region R30 in FIG. 16 displays, on the time axis T, an acquisition time U21 of the report 141, an acquisition time U22 of the report 142, an acquisition time U23 of the report 143, an acquisition time U24 of the report 144, an acquisition time U25 of the report 145, an acquisition time U26 of the report 146, an acquisition time U27 of the report 147, and an acquisition time U28 of the report. 148, so as be associated with the time axis T. In this situation, as illustrated in FIG. 16, the display control function 132 may display the time range in the bar within the region R30.

In this situation, on the display 620, the display control function 132 may present the display in FIG. 16 together with the display of the prescription records in time series or may present the display in FIG. 16 in place of the display of the prescription records in time series. Further, when the medical information processing system 1 includes a plurality of display devices, the display control function 132 may present the display of the prescription records in time series and the display illustrated in FIG. 16 on mutually-different display devices.

The example has so far been explained in which, as the reports corresponding to the time range, the earliest report and the latest report among the reports included in the time range are extracted. However, the extracting function 134 may be configured to extract reports corresponding to the time range by taking the types of the reports into consideration.

For example, at first, the extracting function 134 receives a selecting operation to select the type of reports to be extracted, from the operator. In this situation, examples of the types of reports include image interpretation reports, care providing reports, specimen reports, bacteria reports, physiological reports, and the like. In this situation, the number of types of reports to be selected may be two or more. After that, as the pieces of medical image data corresponding to the time range, the extracting function 134 extracts the earliest report and the latest report from among the reports that are of the selected type and are included in the time range. In another example, as the pieces of medical image data corresponding to the time range, the extracting function 134 extracts, from among the pieces of medical image data that are of the selected types and are included in the time range, the earliest report and the latest report for each of the types of reports. After that, the display control function 132 displays the reports extracted by the extracting function 134 on the display 620.

Further, the example has so far been explained in which, as the pieces of image data corresponding to the time range, either the reports or the pieces of medical image data are extracted. However, the extracting function 134 may extract both the reports and the pieces of medical image data. In other words, as the pieces of first medical data corresponding to the time range, the extracting function 134 is configured to extract at least one selected from between the pieces of medical image data and the reports. For example, at first, the extracting function 134 receives, from the operator, a selecting operation to select the type of image data to be extracted. In this situation, the type of image data may denote a general classification such as “reports” or “medical image data” or may denote a type of reports (e.g., image interpretation reports, care providing reports, specimen reports, bacteria reports, physiological reports, or the like) and a type of medical image data (e.g., X-ray diagnosis apparatuses, X-ray CT apparatuses, MRI apparatuses, ultrasonic diagnosis apparatuses, SPECT apparatuses, PET apparatuses, or the like). Further, the number of types of image data selected in this situation may be two or more.

After that, the extracting function 134 extracts, as the pieces of image data corresponding to the time range, the earliest piece of image data and the latest piece of image data from among the pieces of image data that are of the selected type and are included in the time range. Alternatively, for example, the extracting function 134 extracts, as the pieces of image data corresponding to the time range, the earliest piece of image data and the latest piece of image data for each of the types of image data, from among the pieces of image data that are of the selected types and are included in the time range. In one example, the extracting function 134 extracts the earliest piece of medical image data and the latest piece of medical image data from among the pieces of medical image data included in the time range and further extracts the earliest report and the latest report from among the reports included in the time range. After that, the display control function 132 displays the pieces of medical image data and the reports extracted by the extracting function 134 on the display 620.

In this situation, on the display 620, the display control function 132 may simultaneously present or may switch between the presentations of: the time-series display of the prescription records, the display of the pieces of medical image data extracted by the extracting function 134, and the display of the reports extracted by the extracting function 134. Further, when the medical information processing system 1 includes a plurality of display devices, the display control function may present the following on mutually-different display devices: the time-series display of the prescription records, the display of the pieces of medical image data extracted by the extracting function 134, and the display of the reports extracted by the extracting function 134.

The first to the third embodiments have thus been explained. It is possible to carry out the present disclosure in various different modes other than those in the embodiments described above.

In the embodiments described above, the example is explained in which the prescription records are used as an example of the non-image data; however, possible embodiments are not limited to this example. For instance, as the non-image data, the display control function 132 may display, in time series, numerical value data such as vital data, specimen examination data, image measurement data, or the like, in the same manner as the prescription records are displayed in time series (e.g., the bar graph in FIG. 2D and the shapes in FIG. 10). Further, for example, as the non-image data, the display control function 132 may display text data of medical examination reports, care providing records, or the like in time series, by displaying the text data arranged on a time axis. In other words, as the pieces of second medical data, the display control function 132 is configured to display, in time series, at least one selected from among: prescription records, vital data, specimen examination data, image measurement data, medical examination reports, and care providing records.

In the embodiments described above, the PA server 300, the electronic medical record storage apparatus 400, and the specimen examination server 500 are explained as examples of the memory; however, the memory 120 may function as the memory. In other words, the memory 120 may store therein the pieces of image data indicating medical information and the pieces of non-image data indicating medical information so as to be associated with times.

Further, in the description above, the configuration of the medical information processing system 1 is illustrated in FIG. 1 as an example; however, possible embodiments are not limited to this example. For instance, the medical information processing apparatus 100 included in the medical information processing system 1 may further include an input circuit and a display device (not illustrated). In that situation, the input circuit and the display device included in the medical information processing system 1 have the same functions as those of the input interface 610 and the display 620, so that the medical information processing system 1 does not necessarily have to include the terminal device 600.

Further, in the embodiments described above, the example is explained in which one of the patients is selected; however, two or more of the patients may be selected. For example, at first, the extracting function 134 receives a selecting operation to select the patient P1 and the patient P2 as a plurality of patients. Subsequently, the display control function 132 displays, in time series, pieces of non-image data of at least one selected from between the patient P1 and the patient P2. After that, the acquisition function 133 receives an operation performed on the pieces of non-image data displayed in time series and acquires a time range. The extracting function 134 extracts pieces of image data of the patient P1 corresponding to the time range and pieces of image data of the patient P2 corresponding to the time range. Further, the display control function 132 displays the extracted pieces of image data of the patient P1 and the extracted pieces of image data of the patient P2 on the display 620 or the like.

Further, in the embodiments above, the example is explained in which the operation performed on the pieces of medical image data displayed in time series is received, and the time range corresponding to the received operation is acquired, so that the pieces of non-image data and the time range are displayed in time series while being associated each other. However, possible embodiments are not limited to this example. For instance, at first, the display control function 132 may read the reports stored in the PACS server 300 and display the read reports in time series. After that, the acquisition function 133 receives an operation performed on the reports displayed in time series and acquires a time range corresponding to the received operation. Subsequently, the display control function 132 displays, in time series, the pieces of non-image data and the time range so as to be associated each other.

In the embodiments described above, the example is explained in which the abovementioned processing functions are realized by a single processing circuit (i.e., the processing circuitry 130); however, possible embodiments are not limited to this example. For instance, the processing circuitry 130 may be structured by combining together a plurality of independent processors, so that the processing functions thereof are realized as a result of the processors executing the programs. Further, the processing functions of the processing circuitry 130 may be realized as being distributed to a plurality of processing circuits or being integrated into a single processing circuit, as appropriate.

The term “processor” used in the above explanation denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The one or more processors realize the functions thereof by reading and executing the programs saved in the memory 120. In this situation, instead of saving the programs in the memory 120, it is also acceptable to directly incorporate the programs in the circuits of the processors. In that situation, the processors realize the functions thereof by reading and executing the programs incorporated in the circuits thereof. Further, the processors in the present embodiments do not each necessarily have to be structured as a single circuit. It is also acceptable to structure one processor by combining together a plurality of independent circuits so as to realize the functions thereof.

The programs executed by the one or more processors are provided as being incorporated, in advance, in a Read-Only Memory (ROM), a storage circuit, or the like. Alternatively, the programs may be may be provided as being recorded on a computer-readable storage medium such as a Compact Disk Read-Only Memory (CD-ROM), a flexible disk (ED), a Compact Disk Recordable (CD-R), a Digital Versatile Disk (DVD), or the like, in a file in such a format that is either installable or executable for the devices. Further, the programs may be stored in a computer connected to a network such as the Internet, so as to be provided or distributed as being downloaded via the network. For example, each of the programs is structured with a module including the functions described above. In the actual hardware, as a result of a CPU reading and executing the programs from a storage medium such as a ROM, the modules are loaded into a main storage device so as to be generated in the main storage device.

In the embodiments above, the constituent elements the apparatuses and the devices are based on functional concepts. Thus, it is not necessary to physically configure the constituent elements as indicated in the drawings. In other words, the specific modes of distribution and integration of the apparatuses and the devices are not limited to those illustrated in the drawings. It is acceptable to functionally or physically distribute or integrate all or a part of the apparatuses and the devices in any arbitrary units, depending on various loads and the status of use. Further, all or an arbitrary part of the processing functions performed by the apparatuses and the devices may be realized by a CPU and a program analyzed and executed by the CPU or may be realized as hardware using wired logic.

The processing methods explained in any of the embodiments above may be realized by causing a computer such as a personal computer or a workstation to execute a program prepared in advance. The program may be distributed via a network such as the Internet. Further, the program may be recorded on a computer-readable recording medium such as a hard disk, a flexible disk (FD), a CD-ROM, a Magneto-Optical (MO) disk, a DVD, or the like, so as to be executed as being read from the recording medium by a computer.

According to at least one aspect of the embodiments described above, it is possible to make it easy to compare the first medical data including at least image data, with the second medical data that is included in the data indicating medical information and is other than the first medical data.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing system comprising:
a memory configured to store therein pieces of first medical data each of which includes at least image data indicating medical information and pieces of second medical data each of which is included in data indicating medical information and is other than the first medical data, so that the pieces of first medical data and the pieces of second medical data are associated with times; and
processing circuitry configured to
receive an operation performed on any of the pieces of second medical data displayed in time series, to acquire a time range corresponding to the received operation,
extract at least one of the pieces of first medical data corresponding to the time range from the memory, and
display at least one extracted piece of first medical data.

2. The medical information processing system according to claim 1, wherein the processing circuitry is configured to extract, as at least one of the pieces of first medical data corresponding to the time range, two or more of the pieces of first medical data to be compared with each other that correspond to the time range.

3. The medical information processing system according to claim 1, wherein
the memory is configured to store therein the pieces of first medical data that are of a plurality of types, and
the processing circuitry is configured to extract, as at least one of the pieces of first medical data corresponding to the time range, two or more of the pieces of first medical data to be compared with each other that correspond to the time range, for each of the plurality of types of the first medical data.

4. The medical information processing system according to claim 1, wherein
the memory is configured to store therein the pieces of first medical data that are of a plurality of types, and
the processing circuitry is configured to extract, as at least one of the pieces of first medical data corresponding to the time range, two or more of the pieces of first medical data to be compared with each other that correspond to the time range and are of a type selected from among the plurality of types.

5. The medical information processing system according to claim 2, wherein the processing circuitry is configured to extract, as the pieces of first medical data to be compared with each her, an earliest piece of first medical data and a latest piece of first medical data, from among pieces of first medical data included in the time range.

6. The medical information processing system according claim 1, wherein the processing circuitry is configured to display, as the pieces of second medical data, at least one selected from among: prescription records, vital data, specimen examination data, image measurement data, medical examination reports, and care providing records, in time series.

7. The medical information processing system according to claim 6, wherein the processing circuitry is configured to display, as the prescription records, doses of a selected medication to a selected patient, in time series.

8. The medical information processing system according to claim 1, wherein the processing circuitry is configured to receive, as the operation, a determination operation to determine a first point in time and a second point in time and acquire a time period from the first point in time to the second point in time as the time range.

9. The medical information processing system according to claim 1, wherein the processing circuitry is configured to
receive, as the operation, a determination operation to determine a first point in time and successively acquire a time period from the first point in time to a second point in time that is successively changeable, as the time range,
every time a time range is newly acquired, extract at least one of the pieces of first medical data corresponding to the newly-acquired time range, and
every time at least one of the pieces of first medical data is extracted, display the newly-extracted piece of first medical data.

10. The medical information processing system according to claim 1, wherein the processing circuitry is further configured to display the time range.

11. The medical information processing system according to claim 1, wherein the processing circuitry is further configured to display the pieces of first medical data stored in the memory in thumbnails and also display a bar indicating the times each of which corresponds to a different one of the pieces of first medical data.

12. The medical information processing system according to claim 11, wherein the processing circuitry is further configured to display the time range in the bar.

13. The medical information processing system according to claim 1, wherein the processing circuitry is configured to extract, as at least one of the pieces of first medical data corresponding to the time range, at least one selected from between one or more pieces of medical image data and one or more reports.

14. A medical information processing system comprising:
a memory configured to store therein pieces of first medical data each of which includes at least image data indicating medical information and pieces of second medical data each of which is included in data indicating medical information and is other than the first medical data, so that the pieces of first medical data and the pieces of second medical data are associated with times; and
processing circuitry configured to
receive an operation performed on any of the pieces of first medical data displayed in time series, to acquire a time range corresponding to the received operation, and
display the pieces of second medical data and the time range so as to be associated each other, in time series.

15. The medical information processing system according claim 14, wherein the processing circuitry is configured to
receive, as the operation, a selecting operation to select a first piece of image data and a second piece of image data from among the pieces of first medical data displayed in time series, and
acquire a time period from a time corresponding to the first piece of image data to a time corresponding to the second piece of image data as the time range.

16. The medical information processing system according to claim 14, wherein the processing circuitry is configured to
display, as the pieces of second medical data, at least one selected from among: prescription records, vital data, specimen examination data, image measurement data, medical examination reports, and care providing records, in time series, and
display the time range so as to be associated with the pieces of second medical data displayed in time series.

17. The medical information processing system according to claim 16, wherein the processing circuitry is configured to display, as the prescription records, doses of a selected medication to a selected patient, in time series, and
display the time range so as to be associated with the doses displayed in time series.

18. The medical information processing system according to claim 14, wherein the processing circuitry is configured to display, as the pieces of first medical data, at least one selected from between pieces of medical image data and reports, in time series.
